(19) United States
(12) Patent Application Publication
(10) Pub. No.: US 2025/0369027 A1
(43) Pub. Date: Dec. 4, 2025

(54) SUBSTRATE CLEAVAGE FOR NUCLEIC ACID SYNTHESIS

(71) Applicant: Twist Bioscience Corporation, South San Francisco, CA (US)

(72) Inventors: Jeremy LACKEY, Foster City, CA (US); David DODD, San Francisco, CA (US); Heera LANGNER, San Francisco, CA (US)

(21) Appl. No.: 18/854,437

(22) PCT Filed: Apr. 6, 2023
(86) PCT No.: PCT/US2023/017736

§ 371 Date: Oct. 4, 2024

Related U.S. Application Data
(60) Provisional application No. 63/328,688, filed on Apr. 7, 2022.
(60) Provisional application No. 63/479,672, filed on Jan. 12, 2023.

(51) Int. Cl. C12P 19/34; C12N 9/22; C12N 9/24

(52) U.S. Cl. CPC C12P 19/34; C12N 9/22; C12N 9/2497

(57) ABSTRACT

Disclosed herein are methods and compositions for cleavage of nucleic acids from a surface of a solid support. Further described herein are cleavage methods compatible with enzymatic and chemical nucleic acid synthesis methods.

### CROSS-REFERENCE

This application claims the benefit of U.S. Provisional Application No. 63/328,688 filed Apr. 7, 2022, and U.S. Provisional Application No. 63/479,672 filed Jan. 12, 2023, which are incorporated by reference in their entirety.

### BACKGROUND

Biomolecule based information storage systems, e.g., DNA-based, have a large storage capacity and stability over time. However, there is a need for scalable, automated, highly accurate and highly efficient systems for generating biomolecules for information storage.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Provided herein are methods for cleaving a polynucleotide, comprising: (a) synthesizing a plurality of polynucleotides each comprising one or more bases susceptible to enzymatic cleavage; (b) exposing the plurality of polynucleotides to one or more enzymes; and (c) treating the plurality of polynucleotides in an aqueous base at a temperature of about 55 degrees Celsius to 75 degrees Celsius. In some instances, exposing the plurality of polynucleotides to the one or more enzymes comprises exposing the plurality of polynucleotides to a first enzyme of the one or more enzymes. In some instances, exposing the plurality of polynucleotides to the one or more enzymes further comprises exposing the plurality of polynucleotides to a second enzyme of the one or more enzymes. In some instances, the first enzyme and the second enzyme are different enzymes. In some instances, synthesizing comprises enzymatic synthesis or chemical synthesis. In some instances, synthesizing comprises synthesizing the plurality of polynucleotides on a solid support. In some instances, the plurality of polynucleotides are attached to a surface of the solid support via a support linker. In some instances, the support linker comprises a stilt. In some instances, the stilt comprises thymidine. In some instances, the one or more bases comprises deoxy uracil. In some instances, the one or more enzymes comprises one or more of uracil DNA glycosylase, apurinic/apyrimidinic (AP) endonuclease, alkylpurine glycosylases C and D, OGG1, NTH1, NEIL1-3, Endonuclease V, or endonuclease VII. In some instances, the plurality of polynucleotides are treated in the aqueous base for about one hour. In some instances, the temperature is about 65 degrees Celsius. In some instances, the plurality of polynucleotides encode digital information. In some instances, the digital information comprises text, audio, or visual information.

Further provided herein are methods for cleaving a polynucleotide, comprising: (a) synthesizing a plurality of polynucleotides on a surface of a solid support, wherein the plurality of polynucleotides are attached to the surface via a support linker; and (b) irradiating the plurality of polynucleotides. In some instances, synthesizing comprises enzymatic synthesis or chemical synthesis. In some instances, the support linker comprises a stilt. In some instances, the stilt comprises thymidine. In some instances, the support linker comprises photo-cleavable linker. In some instances, the photo-cleavable linker comprises an orthonitrobenzyl-based linker, phenacyl linker, alkoxybenzoin linker, chromium arene complex linker, NpSSMpact linker, or pivaloylglycol linker. In some instances, the photo-cleavable linker is cleaved by irradiating the support linker at about 312 nm, 365 nm or 405 nm. In some instances, the photo-cleavable linker is irradiated for about 1 minutes to about 15 minutes. In some instances, the plurality of polynucleotides encode digital information. In some instances, the digital information comprises text, audio, or visual information.

Provided herein are methods for synthesizing a polynucleotide, comprising:
a) contacting a polynucleotide with a complex according to the following formula:

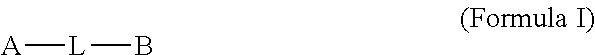

wherein:
A comprises a polymerase;
B comprises a nucleotide; and
L comprises a chemical linker that covalently links the polymerase to a terminal phosphate group of the nucleotide, wherein the polymerase is configured to catalyze covalent addition of the nucleotide onto a 3′ hydroxyl of a polynucleotide, and subsequent extension of the polynucleotide from a surface of a solid support, wherein the polynucleotide is attached to the surface via a support linker; and (b) extending the polynucleotide by addition of the nucleotide, wherein the addition of the nucleotide results in cleavage between the chemical linker and the nucleotide; and (c) cleaving the polymerase from the polynucleotide, wherein the cleaving does not leave a part of the linker on the polynucleotide. Further provided herein are methods wherein the method further comprises cleaving the polynucleotide from the solid support. In some instances, the method further comprises cleaving the polynucleotide from the solid support using a chemical reaction. In some instances, cleavage of the polynucleotide is independently addressable. In some instances, the chemical reaction comprises acid, base, or electrochemistry. In some instances, the method further comprises generation of acid at a region of the surface. In some instances, the acid is generated by applying a potential to a solution containing a mixture of benzoquinone and hydroquinone, or derivatives thereof. In some instances, the support linker comprises an aldol, tetrahydrofuran, or trityl group. In some instances, the method further comprises generation of base at a region of the surface. In some instances, the base is generated by applying a potential to a solution containing (1) an arene or heteroarene; and (2) a protic solvent. In some instances, the arene or heteroarene comprises one or more of substituted or unsubstituted azobenzene, hydrabenzene, azophenanthrene, azonapthalene, and azopyridine. In some instances, the protic solvent comprises an alcohol. In some instances, the base is generated by applying a potential to a solution containing unsubstituted, 1,6 or 2,7 disubstituted phenazine, or tetrasubstituted phenazine with their respective corresponding hydrophenazine compounds. In some instances, the arene or heteroarene comprises a phenolic, cresolic or catecholic group. In some instances, the arene or heteroarene comprises an amine. In some instances, the arene or heteroarene is substituted with one or more of trifluoromethylsulfonyl, hexafluoropropyl, trifluoromethyl, pentafluorophenyl and nitrophenyl. In some instances, the arene or heteroarene is substituted with one or more halogens. In some instances, the support linker comprises an ester. In some instances, the support linker is cleaved by beta elimination. In some instances, the support linker comprises an electron withdrawing group. In some instances, the electron withdrawing group comprises sulfone, fluorine(s), nitro group, sulfonyl or cyano. In some instances, the support linker comprises a latent nucleophile. In some instances, the support linker comprises a levulinyl group. In some instances, the support linker comprises hydroquinone-O,O-diacetic acid (Q-linker). In some instances, the support linker comprises an alkyl-substituted silane. In some instances, the method further comprises an electrochemical reaction. In some instances, the support linker comprise a redox-active group. In some instances, the support linker comprises a metal center. In some instances, the metal center comprises a metal of any one of groups 8-10 of the periodic table. In some instances, the support linker comprises an organoborane. In some instances, the support linker comprises an aryl or alkyl sulfonate. In some instances, the support linker comprises a ligand. In some instances, the support comprises a ligand binder. In some instances, the method comprises cleaving the polynucleotide from the solid support with an enzyme. In some instances, the support linker comprises a stilt. In some instances, the stilt comprises thymidine. In some instances, the support linker comprises uracil. In some instances, the support linker comprises one or more of 3-methyladenine, 8-oxo-guanine, oxo-inosine, 2,6-diamino-4-hydroxy-5-formamidopyrimidine (FapyG), 4,6-diamino-5-formamidopyrimidine (FapyA), 5-hydroxyuracil, 5-hydroxymethyluracil, and 5-formyluracil. In some instances, the enzyme comprises one or more of uracil DNA glycosylase, apurinic/apyrimidinic (AP) endonuclease, alkylpurine glycosylases C and D, OGG1, NTH1, NEIL1-3, Endonuclease V, or endonuclease VII. In some instances, the method further comprises treating the polynucleotide with an aqueous base, heating the polynucleotides, or a combination thereof. In some instances, heating the polynucleotides comprises heating at a temperature of about 55 to 75 degrees Celsius. In some instances, the support linker comprises one or more ribonucleosides. In some instances, the one or more ribonucleosides comprise protecting groups at one or both of the 2′ and 3′ OH positions. In some instances, the protecting groups comprise acetyl, benzoyl, trimethylsilyl, TBDMS, TOM, or levulinyl. In some instances, the enzyme comprises RNase H. In some instances, the method further comprises hybridizing a complementary or partially complementary polynucleotide to the support linker. In some instances, the enzyme comprises one or more of thymidine DNA glycosylase (TDG) and methyl-CpG-binding domain protein 4 (MBD4). In some instances, the enzyme comprises one or more of BamHI, EcoRI, EcoRV, HindIII, and HaeIII. In some instances, steps a)-c) are repeated to produce an extended polynucleotide. In some instances, the extended polynucleotide comprises at least about 10 nucleotides. In some instances, the polymerase is a template-independent polymerase. In some instances, the polymerase is terminal deoxynucleotidyl transferase (TdT) or polymerase theta. In some instances, the chemical linker is an acid-labile linker, a base-labile linker, a pH-sensitive linker, an amine-to-thiol crosslinker, thiomaleamic acid linker, or a photo-cleavable linker. In some instances, the photo-cleavable linker is selected from the group consisting of orthonitrobenzyl-based linker, phenacyl linker, alkoxybenzoin linker, chromium arene complex linker, NpSSMpact linker, pivaloylglycol linker, and any combination thereof. In some instances, the chemical linker is selected from the group consisting of a silyl linker, an alkyl linker, a polyether linker, a polysulfonyl linker, a polysulfoxide linker, and any combination thereof. In some instances, the nucleotide comprises at least 3 phosphate groups. In some instances, the nucleotide is selected from the group consisting of nucleoside triphosphate, nucleoside tetraphosphate, nucleoside pentaphosphate, nucleoside hexaphosphate, nucleoside heptaphosphate, nucleoside octaphosphate, nucleoside nonaphosphate and any combination thereof. In some instances, the nucleotide is selected from the group consisting of deoxyadenosine triphosphate (dATP), deoxyguanosine triphosphate (dGTP), deoxycytidine triphosphate (dCTP), deoxythymidine triphosphate (dTTP), deoxyadenosine tetraphosphate, deoxyguanosine tetraphosphate, deoxycytidine tetraphosphate, deoxythymidine tetraphosphate, deoxyadenosine pentaphosphate, deoxyguanosine pentaphosphate, deoxycytidine pentaphosphate, deoxythymidine pentaphosphate, deoxyadenosine hexaphosphate, deoxyguanosine hexaphosphate, deoxycytidine hexaphosphate, deoxythymidine hexaphosphate, and any combination thereof. In some instances, the polynucleotide encodes digital information. In some instances, the digital information comprises text, audio, or visual information.

Provided herein are methods of synthesizing a polynucleotide, comprising:
(a) contacting a polynucleotide with a complex according to the following formula:

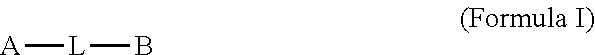

wherein:
A comprises a polymerase;
B comprises a nucleotide; and
L comprises a chemical linker that covalently links the polymerase to a terminal phosphate group of the nucleotide, wherein the polymerase is configured to catalyze covalent addition of the nucleotide onto a 3′ hydroxyl of a polynucleotide, and subsequent extension of the polynucleotide from a surface of a solid support, wherein the polynucleotide is attached to the surface via a support linker; and (b) cleaving the polymerase from the polynucleotide, wherein the cleaving does not leave a part of the linker on the polynucleotide. Further provided herein are methods wherein the method further comprises cleaving the polynucleotide from the solid support. Further provided herein are methods wherein the method further comprises cleaving the polynucleotide from the solid support with an enzyme. Further provided herein are methods, wherein the support linker comprises a stilt. Further provided herein are methods wherein the stilt comprises thymidine. Further provided herein are methods wherein the support linker comprises uracil. Further provided herein are methods wherein the support linker comprises one or more of 3-methyladenine, 8-oxo-guanine, oxo-inosine, 2,6-diamino-4-hydroxy-5-formamidopyrimidine (FapyG), 4,6-diamino-5-formamidopyrimidine (FapyA), 5-hydroxyuracil, 5-hydroxymethyluracil, and 5-formyluracil. Further provided herein are methods wherein the enzyme comprises one or more of uracil DNA glycosylase, apurinic/apyrimidinic (AP) endonuclease, alkylpurine glycosylases C and D, OGG1, NTH1, NEIL1-3, and Endonuclease V. Further provided herein are methods wherein the support linker comprises one or more ribonucleosides. Further provided herein are methods wherein the one or more ribonucleosides comprise protecting groups at one or both of the 2′ and 3′ OH positions. Further provided herein are methods wherein the protecting groups comprise acetyl, benzoyl, trimethylsilyl, TBDMS, TOM, or levulinyl. Further provided herein are methods wherein the enzyme comprises RNase H. Further provided herein are methods wherein the method further comprises hybridizing a complementary or partially complementary polynucleotide to the support linker. Further provided herein are methods wherein the enzyme comprises one or more of thymidine DNA glycosylase (TDG) and methyl-CpG-binding domain protein 4 (MBD4). Further provided herein are methods wherein the enzyme comprises one or more of BamHI, EcoRI, EcoRV, HindIII, and HaeIII. Further provided herein are methods wherein steps a)-b) are repeated to produce an extended polynucleotide. Further provided herein are methods wherein the extended polynucleotide comprises at least about 10 nucleotides. Further provided herein are methods wherein the polymerase is a template-independent polymerase. Further provided herein are methods wherein the polymerase is terminal deoxynucleotidyl transferase (TdT) or polymerase theta. Further provided herein are methods wherein the chemical linker is an acid-labile linker, a base-labile linker, a pH-sensitive linker, an amine-to-thiol crosslinker, thiomaleamic acid linker, or a photo-cleavable linker. Further provided herein are methods wherein the photo-cleavable linker is selected from the group consisting of orthonitrobenzyl-based linker, phenacyl linker, alkoxybenzoin linker, chromium arene complex linker, NpSSMpact linker, pivaloylglycol linker, and any combination thereof. Further provided herein are methods wherein the chemical linker is selected from the group consisting of a silyl linker, an alkyl linker, a polyether linker, a polysulfonyl linker, a polysulfoxide linker, and any combination thereof. Further provided herein are methods wherein the nucleotide comprises at least 3 phosphate groups. Further provided herein are methods wherein the nucleotide is selected from the group consisting of nucleoside triphosphate, nucleoside tetraphosphate, nucleoside pentaphosphate, nucleoside hexaphosphate, nucleoside heptaphosphate, nucleoside octaphosphate, nucleoside nonaphosphate and any combination thereof. Further provided herein are methods wherein the nucleotide is selected from the group consisting of deoxyadenosine triphosphate (dATP), deoxyguanosine triphosphate (dGTP), deoxycytidine triphosphate (dCTP), deoxythymidine triphosphate (dTTP), deoxyadenosine tetraphosphate, deoxyguanosine tetraphosphate, deoxycytidine tetraphosphate, deoxythymidine tetraphosphate, deoxyadenosine pentaphosphate, deoxyguanosine pentaphosphate, deoxycytidine pentaphosphate, deoxythymidine pentaphosphate, deoxyadenosine hexaphosphate, deoxyguanosine hexaphosphate, deoxycytidine hexaphosphate, deoxythymidine hexaphosphate, and any combination thereof. In some instances, the polynucleotide encodes digital information. In some instances, the digital information comprises text, audio, or visual information.

### BRIEF DESCRIPTION OF THE DRAWINGS

FIG. 1A illustrates a first exemplary scheme for cleavage of a support linker to release a nucleic acid bound to a surface. Deprotection of the anomeric hydroxyl group results in opening of the ribose ring, followed by beta elimination to release the polynucleotide.

FIG. 1B illustrates a second exemplary scheme for cleavage of a support linker to release a nucleic acid bound to a surface. Cyclophosphate formation to the 2′ OH displaces 5′ OH of the polynucleotide leading to release of a polynucleotide.

FIG. 2A illustrates addition of an uracil phosphoramidite to a thymine stilt attached to a surface. After enzymatic synthesis steps to add additional bases, enzyme(s) are used to cleave the synthesized polynucleotides from the surface.

FIG. 2B illustrates addition of a protected ribonucleic acid to a thymine stilt attached to a surface. After enzymatic synthesis steps to add additional bases, enzymes (e.g., base or RNase) are used to cleave the synthesized polynucleotides from the surface.

FIG. 3 illustrates an exemplary workflow for nucleic acid-based information storage, according to some embodiments.

FIG. 4 illustrates an example of a computer system, according to some embodiments.

FIG. 5 is a block diagram illustrating an architecture of a computer system, according to some embodiments.

FIG. 6 is a diagram demonstrating a network configured to incorporate a plurality of computer systems, a plurality of cell phones and personal data assistants, and Network Attached Storage (NAS).

FIG. 7 is a block diagram of a multiprocessor computer system using a shared virtual address memory space, according to some embodiments.

FIG. 8A illustrates an exemplary mechanism of enzymatic cleavage of a polynucleotide, according to some embodiments. In some instances, a polynucleotide (A) contains a deoxy uracil that can be cleaved using uracil deglycosylase (B), followed by endonuclease VIII (C). In some instances, exposure of the polynucleotide to one or more enzymes is followed by treatment of aqueous base, heating, or both (C).

FIG. 8B illustrates exemplary LCMS chromatograms from the process illustrated in FIG. 8A, according to some embodiments. The exposure to a polynucleotide (A) to uracil deglycosylase and endonuclease VIII can result in a combination of products B and C, as shown in FIG. 8A (FIG. 8B, top). The top chromatogram shows response units versus acquisition time in minutes. Subsequent treatment with an aqueous base and heat can increase the yield of product C (FIG. 8B, bottom). The bottom chromatograms shows intensity versus time in minutes.

FIG. 9A illustrates an exemplary mechanism of cleavage of a photo-labile linker on a polynucleotide, according to some embodiments. In some embodiments, the photo-labile linker is an orthonitrobenzyl-based linker that can be cleaved by irradiation at a wavelength of about 365 nm.

FIGS. 9B-9C illustrates exemplary LCMS chromatograms for various exposure times of the polynucleotide illustrated in FIG. 9A to irradiation, according to some embodiments. Chromatograms are shown for exposure times of 3 minutes (FIG. 9B, top), 5 minutes (FIG. 9B, bottom), 10 minutes (FIG. 9C, top), and 15 minutes (FIG. 9C, bottom). Each of the chromatograms shown in FIGS. 9B-9C illustrate the response versus acquisition time in minutes.

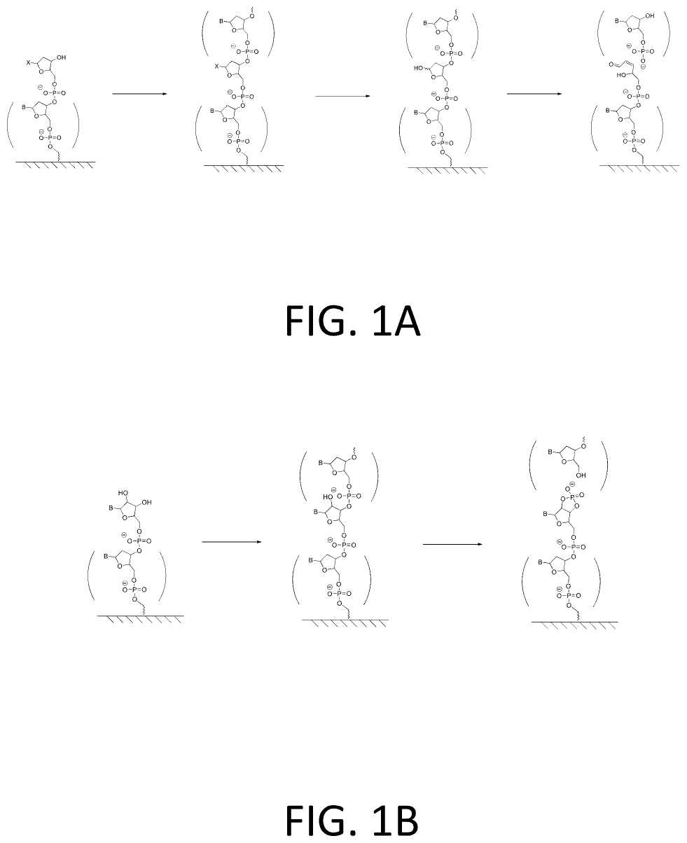

### DETAILED DESCRIPTION

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the term “symbol,” generally refers to a representation of a unit of digital information. Digital information may be divided or translated into one or more symbols. In an example, a symbol may be a bit and the bit may have a numerical value. In some examples, a symbol may have a value of ‘0’ or ‘1’. In some examples, digital information may be represented as a sequence of symbols or a string of symbols. In some examples, the sequence of symbols or the string of symbols may comprise binary data.

Unless specifically stated, as used herein, the term “nucleic acid” encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in 5′ to 3′ direction, unless stated otherwise. Methods described herein provide for the generation of isolated nucleic acids. Methods described herein additionally provide for the generation of isolated and purified nucleic acids. A “nucleic acid” as referred to herein can comprise at least 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more bases in length. Moreover, provided herein are methods for the synthesis of any number of polypeptide-segments encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred herein may comprise at least one region encoding for exon sequences without an intervening intron sequence in the genomic equivalent sequence. cDNA described herein may be generated by de novo synthesis.

Provided herein are methods and compositions for production of polynucleotides. Also provided herein are methods and compositions for cleaving or removing polynucleotides.

Polynucleotides may also be referred to as oligonucleotides or oligos.

### Polynucleotide Synthesis

Polynucleotide synthesis often takes place on a surface of a substrate, such as at discrete loci. After synthesis is completed, polynucleotides are often cleaved from the surface of the substrate. However, cleavage methods often suffer from challenges such as poor yield, harsh conditions/reagents, or damage to newly synthesized polynucleotides. In addition, a multitude of sequences can be synthesized on devices that are too small to independently cleave polynucleotides by chemical means. This can result in complicated analysis and lead to mixed oligo pools if all the synthesized sequences are cleaved at once.

Provided herein are compositions and methods that allow for cleavage of polynucleotides from a substrate. In some instances, the compositions and methods that allow for cleavage of polynucleotides independently from a substrate. Independent cleavage of polynucleotides from a substrate may be performed on a surface comprising addressable loci. Independently cleaving polynucleotides can allow access to certain sequences for different applications (e.g., access to different gene fragments) from a same chip. In some instances, these methods are used in conjunction with chemical or enzymatic polynucleotide synthesis. Polynucleotides, in some instances, are attached to the surface of a substrate or a solid support via a linker. The linker may be referred to as a support linker. In some instances, methods and compositions provided herein cleave the support linker to release the polynucleotides. In some instances, the polynucleotides are released into solution. In some instances, chemical or enzymatic methods are used to cleave a support linker. In some instances, electrochemical methods are used to cleave a support linker (e.g., acid generation).

Provided herein are compositions and methods for improved cleavage of polynucleotides from a surface. In some instances, these methods are used in conjunction with chemical or enzymatic polynucleotide synthesis. Polynucleotides, in some instances, are attached to the surface of a substrate or solid support via a support linker. In some instances, methods and compositions provided herein cleave the support linker to release the polynucleotides into solution. In some instances, chemical or enzymatic methods are used to cleave a support linker. In some instances, enzymatic methods used to cleave a support linker comprise exposure of the support linker to one or more enzymes (e.g., at least one, two, or three enzymes). Exposure of the support linker to one or more enzymes may be performed sequentially.

Provided herein are compositions and methods in which a polynucleotide is attached to a surface via a support linker. In some instances, the support linker comprises a stilt. In some instances, the stilt comprises one or more thymidine. In some instances, the stilt comprises 1-10 thymidine. In some instances, 3′ end of the stilt is attached to a uracil. In some instances, the desired sequence is synthesized enzymatically from the uracil. In some instances, the synthesized polynucleotide is treated with uracil DNA glycosylase which excises the base, leaving an aldehydic anomeric carbon. In some instances, the resulting sugar is then treated with mild base to break the strand leaving 5′ and 3′ phosphate strands. Alternatively, after base excision, treatment with an apurinic/apyrimidinic (AP) endonuclease cleaves the strand. AP classes I-IV in some instances are used to generate alternately phosphorylated or unphosphorylated 3′- and 5′-ends of the cleaved strands.

Base excision repair (BER) enzymes may be used for different endogenous targets. In some instances, a support linker comprises one or more bases configured for removal with a BER. In some instances, a support linker comprises one or more of 3-methyladenine, 8-oxo-guanine, oxo-inosine, 2,6-diamino-4-hydroxy-5-formamidopyrimidine (FapyG), 4,6-diamino-5-formamidopyrimidine (FapyA), 5-hydroxyuracil, 5-hydroxymethyluracil, and 5-formyluracil. These bases in some instances are incorporated using phosphoramidite chemistry with phosphoramidites that contain labile base-protecting groups that may be cleaved before enzymatic synthesis begins. In some instances, alkylpurines are additionally excised by alkylpurine glycosylases C and D (AlkC, AlkD). In some instances, bi-functional DNA glycosylases are used. In some instances, bifunctional glycosylases comprise OGG1, NTH1, NEIL1-3, and their homologues. In some instances, use of bifunctional glycosylases results in no need for a secondary enzymatic treatment. In some instances, a support linker comprises inosine. In some instances, Endonuclease V is used to cleave at an inserted inosine. In some instances, uracil deglycosylase is used to cleave at an inserted inosine. In some examples, uracil deglycosylase followed by endonuclease VII is used to cleave at an inserted inosine.

In some instances, exposure of a polynucleotide to one or more enzymes is followed by treatment with an aqueous base and/or heat for a given time. In some examples, the aqueous base is NH₃/CH₃NH₂. In some examples, the given time is about one hour. In some embodiments, the given time is about 5 minutes, 10 minutes, 15 minutes, 20 minutes, 30 minutes, 45 minutes, 1 hour, 1.5 hours, 2 hours, 3 hours, 4 hours or 5 hours. In some embodiments, the given time is at most about 5 minutes, 10 minutes, 15 minutes, 20 minutes, 30 minutes, 45 minutes, 1 hour, 1.5 hours, 2 hours, 3 hours, 4 hours or 5 hours. In some embodiments, the given time is at least about 5 minutes, 10 minutes, 15 minutes, 20 minutes, 30 minutes, 45 minutes, 1 hour, 1.5 hours, 2 hours, 3 hours, 4 hours or 5 hours. In some embodiments, the given time is about 5-10 minutes, 5-15 minutes, 5-20 minutes, 5-30 minutes, 5 minutes to 1 hour, 10-15 minutes, 10-20 minutes, 10-30 minutes, 10-45 minutes, 10 minutes to 1 hour, 15-20 minutes, 15-30 minutes, 15-45 minutes, 15 minutes to 1 hour, 20-30 minutes, 20-45 minutes, 20 minutes to 1 hour, 30-45 minutes, 30 minutes to 1 hour, 30 minutes to 2 hours, 30 minutes to 3 hours, 45 minutes to 1 hour, 45 minutes to 2 hours, 45 minutes to 3 hours, 1-2 hours, 1-3 hours, 1-4 hours, 1-5 hours, 2-3 hours, 2-4 hours, 2-5 hours, 3-4 hours, 3-5 hours, or 4-5 hours. In some instances, the heat is a temperature of about 30 to 90 degrees Celsius. In some instances, the heat is a temperature of about 55 to 75 degrees Celsius. In some embodiments, the temperature is about 30, 35, 40, 45, 50, 55, 60, 65, 70, 75, 80, 85, or 90 degrees Celsius. In some embodiments, the temperature is at least about 30, 35, 40, 45, 50, 55, 60, 65, 70, 75, 80, 85, or 90 degrees Celsius. In some embodiments, the temperature is at most about 30, 35, 40, 45, 50, 55, 60, 65, 70, 75, 80, 85, or 90 degrees Celsius. In some embodiments, the temperature is about 30-50, 30-60, 30-70, 30-80, 40-60, 40-70, 40-80, 40-90, 45-65, 45-75, 45-85, 50-70, 50-80, 50-90, 55-75, 55-85, 60-80, 60-90, 65-85, or 70-90 degrees Celsius. In some examples, the plurality of polynucleotides are treated in an aqueous base and heated at a temperature for a duration of time (or given time) provided herein.

In some instances, the site where cleavage occurs is further from the start of the enzymatic synthesis. In some instances, the site where cleavage occurs is about 1, 2, 3, 4, 5, 10, 15, 20, 25 or about 30 bases from the start of enzymatic synthesis. In some instances, the site where cleavage occurs is at least 1, 2, 3, 4, 5, 10, 15, 20, 25 or at least 30 bases from the start of enzymatic synthesis. In some instances, the site where cleavage occurs is about 1 to 2, 1 to 3, 1 to 4, 1 to 5, 1 to 10, 1 to 15, 1 to 20, 1 to 25, 1 to 30, 2 to 3, 2 to 4, 2 to 5, 2 to 10, 2 to 15, 2 to 20, 2 to 25, 2 to 30, 3 to 4, 3 to 5, 3 to 10, 3 to 15, 3 to 20, 3 to 25, 3 to 30, 4 to 5, 4 to 10, 4 to 15, 4 to 20, 4 to 25, 4 to 30, 5 to 10, 5 to 15, 5 to 20, 5 to 25, 5 to 30, 10 to 15, 10 to 20, 10 to 25, 10 to 30, 15 to 20, 15 to 25, 15 to 30, 20 to 25, 20 to 30, or 25 to 30 bases from the start of enzymatic synthesis.

An RNA nucleotide may be incorporated into a support linker described herein. In some instances, a support linker comprises an RNA nucleoside at 3′-end of the stilt. In some instances, treatment of this DNA/RNA hybrid with basic conditions results in a 3′-cyclic phosphate at the stilt and a 5′-OH on the enzymatically synthesized strand. In some instances, a complementary strand to the region surrounding the excision site is used for enzymatic cleavage. By hybridization of a DNA complement to the stilt region restriction endonucleases may be used to cleave specific enzymatically synthesized sequences selectively. In some instances, endonucleases comprise BamHI, EcoRI, EcoRV, HindIII, and HaeIII amongst others. In some instances, a partially complementary polynucleotide is used. In some instances, mismatches can also be introduced in this way providing T:G mismatches that are excised by thymidine DNA glycosylase (TDG) and/or methyl-CpG-binding domain protein 4 (MBD4). In some embodiments, several RNA bases may be added to the end of the stilt. In some instances, addition of a DNA complement to the RNA region in the presence of RNase H results in cleavage of the synthesized nucleic acid from the surface. Un-cleaved RNA still present in some instances is later removed enzymatically or through incubation under basic conditions. In some instances, an RNA nucleoside comprises a 5′ protecting group. In some instances, an RNA nucleoside comprises a 3′ protecting group. In some instances, an RNA nucleoside comprises a 3′ and 5′ protecting group. In some instance, the protecting comprises benzoyl, trimethylsilyl, TBDMS, TOM, or levulinyl. In some instance, the protecting is selected from the group consisting of benzoyl, trimethylsilyl, TBDMS, TOM, and levulinyl.

A support linker described herein may comprise nucleotide analogs that are recognized by specific enzymes. In some instances, a support linker comprises a nucleotide analog. In some instances, the support linker comprises deoxy uridine or 8-oxo-deoxyguanosine that are recognized by specific glycosylases (e.g., uracil deoxyglycosylase followed by endonuclease VIII, and 8-oxoguanine DNA glycosylase, respectively). In some embodiments, cleavage by glycosylases and/or endonucleases may require a double stranded DNA substrate. In some embodiments, support linkers comprise base analogs cleavable by endonuclease III which include, but are not limited to, urea, thymine glycol, methyl tartonyl urea, alloxan, uracil glycol, 6-hydroxy-5,6-dihydrocytosine, 5-hydroxyhydantoin, 5-hydroxycytocine, trans-1-carbamoyl-2-oxo-4,5-dihydrooxyimidazolidine, 5,6-dihydrouracil, 5-hydroxy cytosine, 5-hydroxyuracil, 5-hydroxy-6-hydrouracil, 5-hydroxy-6-hydrothymine, 5,6-dihydrothymine. In some embodiments, support linkers comprise base analogs cleavable by formamidopyrimidine DNA glycosylase which include, but are not limited to, 7,8-dihydro-8-oxoguanine, 7,8-dihydro-8-oxoinosine, 7,8-dihydro-8-oxoadenine, 7,8-dihydro-8-oxonebularine, 4,6-diamino-5-formamidopyrimidine, 2,6-diamino-4-hydroxy-5-formamidopyrimidine, 2,6-diamino-4-hydroxy-5-N-methylformamidopyrimidine, 5-hydroxy cytosine, 5-hydroxyuracil. In some embodiments, support linkers comprise base analogs cleavable by hNeil 1 which include, but are not limited to, guanidinohydantoin, spiroiminodihydantoin, 5-hydroxyuracil, thymine glycol. In some embodiments, In some embodiments, support linkers comprise base analogs cleavable by thymine DNA glycosylase which include, but are not limited to, 5-formylcytosine and 5-carboxycytosine. In some embodiments, In some embodiments, support linkers comprise base analogs cleavable by human alkyladenine DNA glycosylase which include, but are not limited to, 3-methyladenine, 3-methylguanine, 7-methylguanine, 7-(2-chloroehyl)-guanine, 7-(2-hydroxyethyl)-guanine, 7-(2-ethoxyethyl)-guanine, 1,2-bis-(7-guanyl) ethane, 1, N⁶-ethenoadenine, 1, N²-ethenoguanine, N²,3-ethenoguanine, N²,3-ethanoguanine, 5-formyluracil, 5-hydroxymethyluracil, hypoxanthine. In some embodiments, support linkers comprise 5-methylcytosine cleavable by 5-methylcytosine DNA glycosylase.

The polynucleotide may be cleaved from a solid support using a chemical reagent. In some embodiments, the support linker is a disulfide bond, which can be cleaved by a reducing agent. In some embodiments, a disulfide support linker is cleaved using β-mercaptoethanol (BME). In some embodiments, the support linker is a base-cleavable bond, such as an ester (e.g., succinate). In some embodiments, the support linker is a base-cleavable linker that can be cleaved, for example, using ammonia or trimethylamine. In some embodiments, the support linker is a quaternary ammonium salt that can be cleaved, for example, using diisopropylamine. In some embodiments, the support linker is a urethane that can be cleaved by a base, such as, for example, aqueous sodium hydroxide.

In some embodiments, the support linker is an acid-cleavable linker. In some embodiments, the support linker is a benzyl alcohol derivative. In some embodiments, the acid-cleavable linker can be cleaved using trifluoroacetic acid. In some embodiments, the support linker teicoplanin aglycone, which can be cleaved by treatment with trifluoroacetic acid and a base. In some embodiments, the support linker is an acetal or thioacetal, which can be cleaved, for example, by trifluoroacetic acid. In some embodiments, the support linker is a thioether that can be cleaved, for example, by hydrogen fluoride or cresol. In some embodiments, the support linker is a sulfonyl group that can be cleaved, for example, by trifluoromethane sulfonic acid, trifluoroacetic acid, or thioanisole. In some embodiments, the support linker comprises a nucleophile-cleavable site, such as a phthalimide that can be cleaved, for example, by treatment with a hydrazine. In some embodiments, the support linker can be an ester that can be cleaved, for example, with aluminum trichloride.

In some embodiments, the support linker is a phosphorothionate that can be cleaved by silver or mercury ions. In some embodiments, the support linker can be a diisopropyldialkoxysilyl group that can be cleaved by fluoride ions. In some embodiments, the support linker can be a diol that can be cleaved by sodium periodate. In some embodiments, the support linker can be an azobenzene that can be cleaved by sodium dithionate.

In some embodiments, the support linker is a photo-cleavable linker. In some embodiments, the photo-cleavable linker is an orthonitrobenzyl-based linker, phenacyl linker, alkoxybenzoin linker, chromium arene complex linker, NpSSMpact linker, or pivaloylglycol linker. In some embodiments, the photo-cleavable linker can be cleaved by irradiating the linker at a wavelength of about 300 to 500 nm. In some embodiments, the photo-cleavable linker can be cleaved by irradiating the linker at about 300 to 400, 300 to 450, 300 to 500, 350 to 370, 350 to 400, 350 to 450, 350 to 500, 400 to 420, 400 to 450, or 400 to 500 nm. In some embodiments, the photo-cleavable linker can be cleaved by irradiating the linker at about 312 nm. In some embodiments, the photo-cleavable linker can be cleaved by irradiating the linker at about 365 nm. In some embodiments, the photo-cleavable linker can be cleaved by irradiating the linker at about 405 nm. In some embodiments, the photo-cleavable linker is irradiated for about 1, 2, 3, 4, 5, 6, 7, 8, 9, or 10 minutes. In some embodiments, the photo-cleavable linker is irradiated for at least about 1, 2, 3, 4, 5, 6, 7, 8, 9, or 10 minutes. In some embodiments, the photo-cleavable linker is irradiated for at most about 1, 2, 3, 4, 5, 6, 7, 8, 9, or 10 minutes. In some embodiments, the photo-cleavable linker is irradiated for about 1-3, 1-5, 1-8, 1-10, 2-4, 2-6, 2-8, 2-10, 3-5, 3-7, 3-9, 3-10, 4-6, 4-8, 4-10, 5-8, 5-10, 6-8, 6-10, 7-9, 7-10, 8-10, or 9-10 minutes.

In some embodiments, the support linker is selected from the group consisting of a silyl linker, an alkyl linker, a polyether linker, a polysulfonyl linker, a polysulfoxide linker, and any combination thereof.

The support-linker may be used to independently cleave one or more polynucleotides from a surface. In some embodiments, the support linker is cleaved by generation of acid at a region of a surface (e.g., electrochemical acid generation). The region can comprise a feature or locus of the solid support. In some embodiments, the region is addressable on the solid support. In some embodiments, the acid is generated by applying a potential to a solution. In some embodiments, the support linker is cleaved by generation of base at a region of a surface. In some embodiments, the support linker is reduced or oxidized to release biomolecules (e.g., polynucleotides) from a region of a surface. In some instances, the surface is a surface of a solid support provided herein.

Acid may be generated by applying a potential to a solution. In some embodiments, the solution comprises a mixture of benzoquinone, and/or hydroquinone, or derivative thereof. In some embodiments, the linker comprises an acid-labile linker. An acid-labile linker may be those provided herein. In some embodiments, the acid-labile linker comprises an aldol, tetrahydrofuran, trityl group, chlorotrityl group, hydroxytrityl group, or other acid labile protecting groups, such as hydrazones, carbonates, cis-aconityl, azidomethyl-methylmaleic anhydride linker, Rink amide linker, FMOC-PAL linker, pyrophosphate linker or any combination thereof.

A linker (e.g., support linker) may be cleaved by a generation of base at a region of a surface. In some instances, the surface is a surface of a solid support provided herein. The region can comprise a feature or locus of the solid support. In some embodiments, the region is addressable on the solid support. Application of a potential to a solution can reverse polarity, which may result in the production of a base when applied to a different solution.

Base may be generated by applying a potential to a solution. In some embodiments, a base is generated using a solution comprising (1) an arene or heteroarene, (2) a protic solvent, or a combination thereof. In some embodiments, the arene or heteroarene comprise a substituted or an unsubstituted azobenzene, hydrabenzene, azophenanthrene, azonapthalene, azopyridine, or any combination thereof. In some embodiments, the solution comprises an azo compound. In some embodiments, the azo compounds comprise aromatic heterocycles. In some embodiments, the solution comprises hydrazo compounds (e.g., hydrazobenzene).

In some embodiments, a base is generated with a solution comprising phenazine. In some embodiments, the phenazine is unsubstituted. In some embodiments, the phenazine is 1,6 or 2,7 disubstituted phenazine. In some embodiments, the phenazine is tetrasubstituted. In some embodiments, the solution comprises a corresponding hydrophenazine compound.

In some embodiments, a protic solvent can comprise an alcohol. In some embodiments, the alcohol is a primary alcohol, secondary alcohol, or tertiary alcohol. In some embodiments, the protonic solvent is deprotonated. In some embodiments, deprotonation of the protic solvent results in a species that can initiate cleavage of biomolecules (e.g., polynucleotides) from a surface of the solid support. In some embodiments, the protic solvent comprises one or more compounds. In some embodiments, the one or more compounds comprises an arene or a heteroarene.

In some embodiments, the arene or heteroarene comprises a phenolic group, cresolic group, catecholic group, or any combination thereof. In some embodiments, the arene or heteroarene comprises an amine. In some embodiments, pKa of the amino proton in the arene or heteroarene is manipulated by a substitution. In some embodiments, the arene or heteroarene is substituted with a trifluoromethylsulfonyl, hexafluoropropyl, trifluoromethyl, pentafluorophenyl, nitrophenyl, or any combination thereof. In some embodiments, the arene or heteroarene is substituted with one or more halogens. In some embodiments, the one or more halogens comprise F, Cl, Br, I, or any combination thereof. In some embodiments, the one or more halogens manipulate the pKa of the compound.

In some embodiments, the linker comprises an ester.

In some embodiments, the linker is cleaved by beta elimination. In some embodiments, the linker is cleaved similar to decyanoethylaton of a phosphate backbone in phosphoramidite chemistry. In some embodiments, the linker comprises an electron withdrawing group (EWG). In some embodiments, the EWG comprises sulfone, fluorine(s), nitro group, sulfonyl, cyano, or any combination thereof.

In some embodiments, the linker comprises a latent nucleophile. In some embodiments, the latent nucleophile produces a nucleophile when activated. In some embodiments, activation of the nucleophile results in self-cleavage of the linker. In some embodiments, activation of the nucleophile results in cleavage of biomolecules (e.g., polynucleotides) from a surface of the solid support.

In some embodiments, the linker comprises a levulinyl group.

In some embodiments, the linker comprises hydroquinone-O,O-diacetic acid (Q-linker).

In some embodiments, the linker comprises an alkyl-substituted silane. In some embodiments, the alkyl-substituted silane is cleaved by electrochemical production of an alkoxide.

A linker may be reduced or oxidized to release biomolecules (e.g., polynucleotides) from a surface of a solid support. In some embodiments, the linker comprises a redox-active group. In some embodiments, the linker comprises a metal center. In some embodiments, the metal center comprises a metal of any one of groups 8-10 of the periodic table. In some embodiments, the metal center is pro-catalytic. In some embodiments, the metal center is ligated. In some embodiments, the metal center is unligated.

In some embodiments, the linker comprises an organoborane. In some embodiments, the linker is cleaved through oxidative elimination followed by reductive elimination. In some embodiments, the linker comprises an aryl, an alkyl sulfonate, or a combination thereof. In some embodiments, the aryl or alkyl sulfonate oxidatively adds to the metal center.

In some embodiments, the linker comprises a ligand. In some embodiments, the linker comprises a transition metal complex. In some embodiments, the transition metal complex undergoes oxidation or reduction. In some embodiments, the oxidation or reduction causes a structural change resulting in the release of a ligand-modified biomolecules (e.g., polynucleotides). In some embodiments, biomolecules are tethered to the surface of a solid support by ligation. In some embodiments, the biomolecules are released via a deprotonation reaction. In some embodiments, the biomolecules are released by demasking a ligand with a lower dissociation constant in respect to the metal center. In some embodiments, a metal center or a complex comprising a metal center is anchored to the surface. In some embodiments, a metal center or a complex comprising a metal center is free floating in a solution.

In some embodiments, the support linker comprises an aldol, tetrahydrofuran, chlorotrityl group, hydroxytrityl group, or other acid labile protecting groups, such as hydrazones, carbonates, cis-aconityl, azidomethyl-methylmaleic anhydride linker, Rink amide linker, FMOC-PAL linker, pyrophosphate linker or any combination thereof. In some embodiments, the support linker comprises an ester. In some embodiments, the support linker is cleaved by beta elimination. In some embodiments, the support linker comprises an electron withdrawing group (EWG). In some embodiments, the EWG comprises sulfone, fluorine(s), nitro group, sulfonyl, cyano, or any combination thereof. In some embodiments, the support linker comprises a latent nucleophile. In some embodiments, the support linker comprises a levulinyl group. In some embodiments, the support linker comprises hydroquinone-O,O-diacetic acid (Q-linker). In some embodiments, the support linker comprises an alkyl-substituted silane. In some embodiments, the alkyl-substituted silane is cleaved by electrochemical production of an alkoxide. In some embodiments, the support linker comprises a redox-active group. In some embodiments, the support linker comprises a metal center. In some embodiments, the metal center comprises a metal of any one of groups 8-10 of the periodic table. In some embodiments, the support linker comprises an organoborane. In some embodiments, the support linker comprises an aryl, an alkyl sulfonate, or a combination thereof. In some embodiments, the linker comprises a ligand.

Enzymes may be used to synthesize polynucleotides. Terminal deoxynucleotidyl transferase (TdT) is a polymerase that adds deoxynucleotide triphosphates (dNTPs) to the 3′ end of single-stranded DNA. Disclosed herein are methods of enzymatically synthesizing polynucleotides using TdT. A two-step method is used to extend polynucleotides using TdT-dNTP conjugates consisting of a TdT molecule site-specifically labeled with a dNTP via a cleavable linker. The synthetic cycle comprises two steps: (1) In the extension step, a DNA primer is exposed to an excess of TdT-dNTP conjugate. Once the tethered nucleotide is incorporated into the 3′ end of the primer, the conjugate becomes covalently attached, which prevents extensions by other TdT-dNTP molecules. Each TdT molecule is conjugated to a single dNTP molecule that is incorporated into a primer. (2) In the deprotection step, the excess TdT-dNTP conjugates are inactivated, and the linkage between the incorporated nucleoside and TdT is cleaved. Cleavage of TdT releases the primer for further extension. The two-step process can be repeated to generate a defined sequence.

Described herein are methods of synthesizing polynucleotides comprising using a complex according to the following formula:

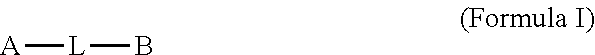

wherein A comprises a polymerase; B comprises a nucleotide; and L comprises a chemical linker that covalently links the polymerase to a terminal phosphate group of the nucleotide, wherein the polymerase is configured to catalyze covalent addition of the nucleotide onto a 3′ hydroxyl of a polynucleotide, and subsequent extension of the polynucleotide. Following extension of the polynucleotide, the polynucleotide may be cleaved using the methods and compositions described herein. In some embodiments, using the compositions and methods described herein, cleaving does not leave a part of the linker on the polynucleotide. In some instances, the chemical linker and the support linker are different.

In some embodiments, the polymerase is site-specifically conjugated to a terminal phosphate group of a phosphorylated nucleoside to form a tethered molecule. A phosphorylated nucleoside, in some embodiments, is referred to as a nucleotide. When a polymerase incorporates the tethered phosphorylated nucleoside into a primer, the polymerase can remain covalently attached to a terminal phosphate group of 3′ end of the primer via a linker, blocking further elongation by other polymerase conjugates. The linker can then be cleaved to deprotect 3′ end of the primer for subsequent extension. The process can be repeated to elongate the polynucleotide to a desired length and sequence. In some instances, extending the polynucleotide comprise incorporating the nucleotide. In some instances, incorporating the nucleotide results in spontaneous cleavage between the linker and the nucleotide and release of the polymerase, linker, or both. In some instances, the polymerase is released from the extended polynucleotide after condensation. In some instances, cleavage and release of the polymerase-linker-5P happens spontaneously upon reaction to 3′-end of the polynucleotide.

In some embodiments, the phosphorylated nucleoside (e.g., nucleotide) to be tethered to the polymerase is a nucleoside comprising at least one phosphate group. In some embodiments, the nucleoside comprises at least 1, 2, 3, 4, 5, 6, 7, 8, 9, or more than 9 phosphate groups. In some embodiments, the nucleoside comprises at least 3 phosphate groups. In some embodiments, the phosphorylated nucleoside is adenosine, cytidine, uridine, or guanosine, each of which comprises at least one phosphate group. In some embodiments, the phosphorylated nucleoside is a deoxynucleoside comprising at least one phosphate group. In some embodiments, the phosphorylated nucleoside is a deoxynucleoside comprising at least 3 phosphate groups. In some embodiments, the deoxynucleoside comprises at least 1, 2, 3, 4, 5, 6, 7, 8, 9, or more than 9 phosphate groups. In some embodiments, the phosphorylated nucleoside is deoxyadenosine, deoxycytidine, deoxythymidine, or deoxyguanosine, each of which comprises at least one phosphate group. In some embodiments, the phosphorylated nucleoside is a nucleoside triphosphate, such as dNTP. In some embodiments, the phosphorylated nucleoside is a nucleoside tetraphosphate, nucleoside pentaphosphate, a nucleoside hexaphosphate, a nucleoside heptaphosphate, nucleoside octaphosphate, or a nucleoside nonaphosphate. In some embodiments, the phosphorylated nucleoside is a nucleoside hexaphosphate. In some embodiments, the phosphorylated nucleoside is a nucleoside triphosphate. In some embodiments, the phosphorylated nucleoside is selected from the group consisting of deoxyadenosine triphosphate (dATP), deoxyguanosine triphosphate (dGTP), deoxycytidine triphosphate (dCTP), deoxythymidine triphosphate (dTTP), deoxyadenosine tetraphosphate, deoxyguanosine tetraphosphate, deoxycytidine tetraphosphate, deoxythymidine tetraphosphate, deoxyadenosine pentaphosphate, deoxyguanosine pentaphosphate, deoxycytidine pentaphosphate, deoxythymidine pentaphosphate, deoxyadenosine hexaphosphate, deoxyguanosine hexaphosphate, deoxycytidine hexaphosphate, deoxythymidine hexaphosphate, and any combination thereof.

The methods described herein can use enzymatically synthesized polynucleotides using a solid support. In some embodiments, the methods of the disclosure can synthesize polynucleotides in the wells of a multi-well plate, for example, 96-well or 384-well plates. In some embodiments, the methods of the disclosure can synthesize polynucleotides using a non-swellable or low-swellable solid support. In some embodiments, the methods of the disclosure can synthesize polynucleotides using controlled pore glass (CPG) or microporous polystyrene (MPPS). In some embodiments, the methods of the disclosure can synthesize polynucleotides on CPG treated with a surface-coating material. In some embodiments, the methods of the disclosure can synthesize polynucleotides on CPG treated with (3-aminopropyl)triethoxysilane (3-aminopropyl CPG). In some embodiments, the methods of the disclosure can synthesize polynucleotides on long chain aminoalkyl (LCAA) CPG. In some embodiments, the methods of the disclosure can synthesize polynucleotides using CPG with average pore sizes of about 500, about 1000, about 1500, about 2000, or about 3000 Å.

Provided herein are various surfaces for enzymatically synthesized polynucleotides. In some embodiments, the surface comprises one or more reverse phosphoramidites. In some embodiments, the surface comprises a linker attached on the surface. In some embodiments, the linker is attached on the surface after treatment with diethylamine. In some embodiments, the surface comprises dT.

In some embodiments, the surface comprises at least one hydrophilic polymer. The hydrophilic polymer comprises, in various embodiments, polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo (ethylene glycol) methyl ether methacrylate) (POEGMA), polyglutamic acid (PGA), poly-lysine, poly-glucoside, streptavidin, and dextran. In some embodiments, the surface comprises polyethylene glycol (PEG).

In some embodiments, the surface comprises a siloxane monomer or polymer. In some embodiments, the siloxane monomer or polymer comprises an epoxide functional group. In some embodiments, the siloxane monomer or polymer thereof comprises one or more monomers selected from (3-glycidylpropyl) trimethoxysilane (GPTMS), Diethoxy (3-glycidyloxypropyl)methylsilane, 3-Glycidoxypropyldimethoxymethylsilane, 2-(3,4-epoxycyclohexyl)ethyltriethoxysilane, 2-(3,4-epoxycyclohexyl)ethyltrimethoxysilane, or combinations thereof. In some embodiments, the siloxane monomer is GPTMS. In some embodiments, the siloxane monomer is Diethoxy (3-glycidyloxypropyl)methylsilane. In some embodiments, the siloxane monomer is 3-Glycidoxypropyldimethoxymethylsilane. In some embodiments, the siloxane monomer is 2-(3,4-epoxycyclohexyl)ethyltriethoxysilane. In some embodiments, the siloxane monomer is 2-(3,4-epoxycyclohexyl)ethyltrimethoxysilane.

In some embodiments, the surfaces comprise heptadecafluorodecyltrichlorosilane, poly(tetrafluoroethylene), octadecyltrichlorosilane, methyltrimethoxysilane, nonafluorohexyltrimethoxysilane, vinyltriethoxysilane, paraffin wax, ethyltrimethoxysilane, propyltrimethoxysilane, glass, poly(chlorotrifluoroethylene), polypropylene, poly(propylene oxide), polyethylene, trifluoropropyltrimethoxysilane, 3-(2-aminoethyl)aminopropyltrimethoxysilane, polystyrene, p-tolyltrimethoxysilane, cyanoethyltrimethoxysilane, aminopropyltriethoxysilane, acetoxypropyltrimethoxysilane, poly(methyl methacrylate), poly(vinyl chloride), phenyltrimethoxysilane, chloropropyltrimethoxysilane, mercaptopropyltrimethoxysilane, glycidoxypropyltrimethoxysilane, poly(ethylene terephthalate), copper (dry), poly(ethylene oxide), aluminum, nylon 6/6, iron (dry), glass, sodalime (dry), titanium oxide (anatase), ferric oxide, tin oxide, or combinations thereof.

Provided herein are various supports for enzymatically synthesized polynucleotides. In some embodiments, the polynucleotides described herein are synthesized on one or more solid supports. Exemplary solid supports include, for example, slides, beads, chips, particles, strands, gels, sheets, tubing, spheres, containers, capillaries, pads, slices, films, plates, polymers, or a microfluidic device. Further, the solid supports may be biological, nonbiological, organic, inorganic, or combinations thereof. On supports that are substantially planar, the support may be physically separated into regions, for example, with trenches, grooves, wells, or chemical barriers (e.g., hydrophobic coatings, etc.). Supports may also comprise physically separated regions built into a surface, optionally spanning the entire width of the surface. Suitable supports for improved oligonucleotide synthesis are further described herein. In some embodiments, the polynucleotides are provided on a solid support for use in a microfluidic device, for example, as part of the PCA reaction chamber. In some embodiments, the polynucleotides are synthesized and subsequently introduced into a microfluidic device. In some embodiments, the solid support is part of or is integrated into a flow cell assembly.

Provided herein are devices for polynucleotide synthesis. The devices can comprise an addressable solid support for independent cleavage of one or more polynucleotides. In some instances, the device may comprise an addressable region or loci in which polynucleotides are synthesized. In some instances, the addressable regions or loci are in fluid communication with solvents and other reagents for polynucleotide synthesis and/or subsequent cleavage of one or more polynucleotides from the solid support.

The solid support for polynucleotide synthesis can comprise a number of sites (e.g., spots) or positions for synthesis. In some instances, the solid supports can be used to polynucleotide storage. In some instances, the solid support comprises up to or about 10,000 by 10,000 positions in an area. In some instances, the solid support comprises between about 1000 and 20,000 by between about 1000 and 20,000 positions in an area. In some instances, the solid support comprises at least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions by least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions in an area. In some instances the area is up to 0.25, 0.5, 0.75, 1.0, 1.25, 1.5, or 2.0 inches squared. In some instances, the solid support comprises addressable loci having a pitch of at least or about 0.1, 0.2, 0.25, 0.3, 0.4, 0.5, 1.0, 1.5, 2.0, 2.5, 3.0, 3.5, 4.0, 4.5, 5, 6, 7, 8, 9, 10, or more than 10 μm. In some instances, the solid support comprises addressable loci having a pitch of about 5 μm. In some instances, the solid support comprises addressable loci having a pitch of about 2 μm. In some instances, the solid support comprises addressable loci having a pitch of about 1 μm. In some instances, the solid support comprises addressable loci having a pitch of about 0.2 μm. In some instances, the solid support comprises addressable loci having a pitch of about 0.2 μm to about 10 μm, about 0.2 to about 8 μm, about 0.5 to about 10 μm, about 1 μm to about 10 μm, about 2 μm to about 8 μm, about 3 μm to about 5 μm, about 1 μm to about 3 μm or about 0.5 μm to about 3 μm. In some instances, the solid support comprises addressable loci having a pitch of about 0.1 μm to about 3 μm. In some instances, the solid support comprises addressable loci having a pitch of at least or about 0.01, 0.02, 0.025, 0.03, 0.04, 0.05, 0.1, 0.15, .02, 0.25, 0.30, 0.35, 0.4, 0.45, 0.5, 0.6, 0.7, 0.8, 0.9, 1, or more than 1 μm. In some instances, the solid support comprises addressable loci having a pitch of about 0.5 μm. In some instances, the solid support comprises addressable loci having a pitch of about 0.2 μm. In some instances, the solid support comprises addressable loci having a pitch of about 0.1 μm. In some instances, the solid support comprises addressable loci having a pitch of about 0.02 μm. In some instances, the solid support comprises addressable loci having a pitch of about 0.02 μm to about 1 μm, about 0.02 to about 0.8 μm, about 0.05 to about 0.1 μm, about 0.1 μm to about 1 μm, about 0.2 μm to about 0.8 μm, about 0.3 μm to about 0.5 μm, about 0.1 μm to about 0.3 μm or about 0.05 μm to about 0.3 μm. In some instances, the solid support comprises addressable loci having a pitch of about 0.01 μm to about 0.3 μm.

Chemical reactions used in polynucleotide synthesis and/or subsequent cleavage of one or more polynucleotides can be controlled using electrochemistry. Electrochemical reactions in some instances are controlled by any source of energy, such as light, heat, radiation, or electricity. For example, electrodes are used to control chemical reactions as all or a portion of discrete loci on a surface. Electrodes in some instances are charged by applying an electrical potential to the electrode to control one or more chemical steps in polynucleotide synthesis. In some instances, these electrodes are addressable. Any number of the chemical steps described herein is in some instances controlled with one or more electrodes. Electrochemical reactions may comprise oxidations, reductions, acid/base chemistry, or other reaction that is controlled by an electrode. In some instances, electrodes generate electrons or protons that are used as reagents for chemical transformations. Electrodes in some instances directly generate a reagent such as an acid. In some instances, an acid is a proton. Electrodes in some instances directly generate a reagent such as a base. Acids or bases are often used to cleave protecting groups, or influence the kinetics of various polynucleotide synthesis reactions, for example by adjusting the pH of a reaction solution. Electrochemically controlled polynucleotide synthesis reactions in some instances comprise redox-active metals or other redox-active organic materials. In some instances, metal or organic catalysts are employed with these electrochemical reactions. In some instances, acids are generated from oxidation of quinones.

Control of chemical reactions can comprise but is not limited to electrochemical generation of reagents; chemical reactivity may be influenced indirectly through biophysical changes to substrates or reagents through electric fields (or gradients) which are generated by electrodes. In some instances, substrates include but are not limited to nucleic acids. In some instances, electrical fields which repel or attract specific reagents or substrates towards or away from an electrode or surface are generated. Such fields in some instances are generated by application of an electrical potential to one or more electrodes. For example, negatively charged nucleic acids are repelled from negatively charged electrode surfaces. Such repulsions or attractions of polynucleotides or other reagents caused by local electric fields in some instances provides for movement of polynucleotides or other reagents in or out of region of the synthesis device or structure. In some instances, electrodes generate electric fields which repel polynucleotides away from a synthesis surface, structure, or device. In some instances, electrodes generate electric fields which attract polynucleotides towards a synthesis surface, structure, or device. In some instances, protons are repelled from a positively charged surface to limit contact of protons with substrates or portions thereof. In some instances, repulsion or attractive forces are used to allow or block entry of reagents or substrates to specific areas of the synthesis surface. In some instances, nucleoside monomers are prevented from contacting a polynucleotide chain by application of an electric field in the vicinity of one or both components. Such arrangements allow gating of specific reagents, which may obviate the need for protecting groups when the concentration or rate of contact between reagents and/or substrates is controlled. In some instances, unprotected nucleoside monomers are used for polynucleotide synthesis. Alternatively, application of the field in the vicinity of one or both components promotes contact of nucleoside monomers with a polynucleotide chain. Additionally, application of electric fields to a substrate can alter the substrates reactivity or conformation. In an exemplary application, electric fields generated by electrodes are used to prevent polynucleotides at adjacent loci from interacting. In some instances, the substrate is a polynucleotide, optionally attached to a surface. Application of an electric field in some instances alters the three-dimensional structure of a polynucleotide. Such alterations comprise folding or unfolding of various structures, such as helices, hairpins, loops, or other 3-dimensional nucleic acid structure. Such alterations are useful for manipulating nucleic acids inside of wells, channels, or other structures. In some instances, electric fields are applied to a nucleic acid substrate to prevent secondary structures. In some instances, electric fields obviate the need for linkers or attachment to a solid support during polynucleotide synthesis.

Conventional methods of electrochemical acid generation often require voltages in excess of those tolerable by high-density transistor devices (e.g., CMOS). Excess voltages in some instances give rise to unstable currents which reduce the fidelity of deblocking during polynucleotide synthesis. In some instances, methods described herein are configured to operate at voltages less than 2 volts. In some instances, methods described herein are configured for voltages of no more than 2.00, 1.95, 1.9, 1.85, 1.80, 1.75, 1.70, 1.65, 1.60, or no more than 1.50 volts. In some instances, methods described herein are configured for voltages of 0.1-2, 0.1-1.5, 1-1.9, 1-1.8, 1-1.7, 1-1.6 or 1-1.5 volts. In some instances, compositions described herein allow for reduced concentrations of redox compounds relative to previous methods. In some instances, compositions described herein allow for reduced concentrations of additives, such as reduced or eliminate concentrations of bases. In some instances, compositions described herein allow for reduced concentrations of additives, such as reduced or eliminate concentrations of amine bases, (e.g., 2,6-lutidine).

Provided herein are devices for enzymatically synthesized polynucleotides comprising layers of materials. Such devices may comprise any number of layers of materials comprising conductors, semiconductors, or insulative materials. Various layers of such devices are in some instances combined to form addressable solid supports. Layers or surfaces of such devices may be in fluid communication with solvents, solutes, or other reagents used during polynucleotide synthesis. Further described herein are devices comprising a plurality of surfaces. In some instances, surfaces comprise features for polynucleotides synthesis in proximity to conducting materials. In some instances, devices described herein comprise 1, 2, 5, 10, 50, 100, or even thousands of surfaces per device. In some instances, a voltage is applied to one or more layers of a device described herein to facilitate polynucleotide synthesis. In some instances, a voltage is applied to one or more layers of a device described herein to facilitate a step in polynucleotide synthesis, such as deblocking. Different layers on different surfaces of different devices are often energized with a voltage at varying times or with varying voltages. For example, a positive voltage is applied to a first layer, and a negative voltage is applied to a second layer of the same or a different device. In some instances, one or more layers on different devices are energized, while others are disconnected from a ground. In some instances, base layers comprise additional circuitry, such as complementary metal-oxide-semiconductors (CMOS) devices. In some instances, various layers of one or more devices are connected laterally via routing, and/or vertically with vias. In some instances, various layers of one or more devices are connected laterally via routing, and/or vertically with vias to a CMOS layer. In some instances, various layers of one or more devices are connected to a CMOS device via wire bonds, pogo pin contacts, or through Si Vias (TSV).

The substrates, the solid support, or the devices described herein may be fabricated from a variety of materials, suitable for the methods and compositions of the disclosure described herein. In certain embodiments, the materials from which the substrates/solid supports of the comprising the disclosure are fabricated exhibit a low level of oligonucleotide binding. In some situations, material that are transparent to visible and/or UV light can be employed. Materials that are sufficiently conductive, e.g. those that can form uniform electric fields across all or a portion of the substrates/solids support described herein, can be utilized. In some embodiments, such materials may be connected to an electric ground. In some cases, the substrate or solid support can be heat conductive or insulated. The materials can be chemical resistant and heat resistant to support chemical or biochemical reactions such as a series of oligonucleotide synthesis reaction. For flexible materials, materials of interest can include: nylon, both modified and unmodified, nitrocellulose, polypropylene, and the like. For rigid materials, specific materials of interest include: glass; fuse silica; silicon, plastics (for example polytetrafluoroethylene, polypropylene, polystyrene, polycarbonate, and blends thereof, and the like); metals (for example, gold, platinum, and the like). The substrate, solid support or reactors can be fabricated from a material selected from the group consisting of silicon, polystyrene, agarose, dextran, cellulosic polymers, polyacrylamides, polydimethylsiloxane (PDMS), and glass.

In various embodiments, surface modifications are employed for the chemical and/or physical alteration of a surface by an additive or subtractive process to change one or more chemical and/or physical properties of a substrate surface or a selected site or region of a substrate surface. For example, surface modification may involve (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

Described herein are methods for enzymatically synthesizing polynucleotides. In some embodiments, the methods comprise using a chain-elongating enzyme. In some instances, the chain-elongating enzyme is a polymerase. In some instances, the polymerase is a template-independent polymerase. In some instances, the polymerase is an RNA polymerase or DNA polymerase. In some instances, the polymerase is a DNA polymerase. Examples of DNA polymerases include polA, polB, polC, polD, polY, polX, reverse transcriptases (RT), and high-fidelity polymerases. In some instances, the polymerase is a modified polymerase.

In some embodiments, the polymerase comprises Φ29, B103, GA-1, PZA, Φ15, BS32, M2Y, Nf, G1, Cp-1, PRD1, PZE, SF5, Cp-5, Cp-7, PR4, PR5, PR722, L17, ThermoSequenase®, 9°Nm™, Therminator™ DNA polymerase, Tne, Tma, TfI, Tth, TIi, Stoffel fragment, Vent™ and Deep Vent™ DNA polymerase, KOD DNA polymerase, Tgo, JDF-3, Pfu, Taq, T7 DNA polymerase, T7 RNA polymerase, PGB-D, UITma DNA polymerase, E. coli DNA polymerase I, E. coli DNA polymerase III, archaeal DP 1I/DP2 DNA polymerase II, 9°N DNA Polymerase, Taq DNA polymerase, Phusion® DNA polymerase, Pfu DNA polymerase, SP6 RNA polymerase, RB69 DNA polymerase, Avian Myeloblastosis Virus (AMV) reverse transcriptase, Moloney Murine Leukemia Virus (MMLV) reverse transcriptase, SuperScript® II reverse transcriptase, and SuperScript® III reverse transcriptase.

In some embodiments, the polymerase is DNA polymerase 1-Klenow fragment, Vent polymerase, Phusion® DNA polymerase, KOD DNA polymerase, Taq polymerase, T7 DNA polymerase, T7 RNA polymerase, Therminator™ DNA polymerase, POLB polymerase, SP6 RNA polymerase, E. coli DNA polymerase I, E. coli DNA polymerase III, Avian Myeloblastosis Virus (AMV) reverse transcriptase, Moloney Murine Leukemia Virus (MMLV) reverse transcriptase, SuperScript® II reverse transcriptase, or SuperScript® III reverse transcriptase.

The polymerase molecules used in the methods described herein can be polymerase theta, a DNA polymerase, or any enzyme that can extend nucleotide chains. In some embodiments, the polymerase is tri29. In some embodiments, the polymerase is a protein with pockets that work around terminal phosphate groups, for example, a triphosphate group.

In some embodiments, the described methods use TdT with 1, 2, 3, 4, 5, 6, 7, 8, 9, or 10 amino acid mutations to synthesize defined polynucleotides. In some embodiments, the described method uses TdT with 1, 2, 3, 4, 5, 6, 7, 8, 9, or 10 amino acid mutations to a surface-accessible amino acid residue. In some embodiments, the TdT is a variant of TdT. In some embodiments, the variant of TdT comprises a cysteine mutation (e.g., NTT-1). In some embodiments, the variant of TdT is NTT-1, NTT-2, or NTT-3. In some instances, the variant TdT comprises at least 70%, 80%, 90%, or 95% sequence identity to wild-type TdT.

In some embodiments, the described methods use polymerase theta with 1, 2, 3, 4, 5, 6, 7, 8, 9, or 10 amino acid mutations to synthesize defined polynucleotides. In some embodiments, the described method uses polymerase theta with 1, 2, 3, 4, 5, 6, 7, 8, 9, or 10 amino acid mutations to a surface-accessible amino acid residue. In some embodiments, the polymerase theta is a variant of polymerase theta. In some instances, the variant polymerase theta comprises at least 70%, 80%, 90%, or 95% sequence identity to wild-type polymerase theta. In some embodiments, the polymerase theta is encoded by POLQ.

Enzymes described herein (e.g., TdT), in some embodiments, comprise one or more unnatural amino acids. In some instances, the unnatural amino acid comprises: a lysine analogue; an aromatic side chain; an azido group; an alkyne group; or an aldehyde or ketone group. In some instances, the unnatural amino acid does not comprise an aromatic side chain. In some embodiments, the unnatural amino acid is selected from N6-azidoethoxy-carbonyl-L-lysine (AzK), N6-propargylethoxy-carbonyl-L-lysine (PraK), N6-(propargyloxy)-carbonyl-L-lysine (PrK), p-azido-phenylalanine (pAzF), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl) alanine, 2-amino-3-((2-((3-(benzyloxy)-3-oxopropyl)amino)ethyl) selanyl) propanoic acid, 2-amino-3-(phenylselanyl) propanoic, selenocysteine, N6-(((2-azidobenzyl)oxy) carbonyl)-L-lysine, N6-(((3-azidobenzyl)oxy) carbonyl)-L-lysine, and N6-(((4-azidobenzyl)oxy) carbonyl)-L-lysine.

In some embodiments, the enzymes described herein are fused to one more other enzymes. For example, TdT is fused to other enzymes such as helicase.

Various linkers are provided herein for conjugating an enzyme or other nucleic acid (e.g., polymerase) binding moiety to one or more base-pairing moieties, e.g., a modified nucleotide during enzymatic synthesis of the polynucleotides. Conjugation of nucleotides or other base-pairing moieties to linkers may be achieved by any means known in the art of chemical conjugation methods. For example, nucleotides containing base modifications that add a free amine group are contemplated for use in conjugation to linkers as described herein. Primary amines, for example, may be linked to the base in such a manner that they can be reacted with heterobifunctional polyethylene glycol (PEG) linkers to create a nucleotide containing a variable length PEG linker that will still bind properly to the enzyme active site. Examples of such amine-containing nucleotides include 5-propargylamino-dNTPs, 5-propargylamino-NTPs, amino allyl-dNTPs, and amino allyl-NTPs.

In some embodiments, amine-containing nucleotides are suitable for conjugation with PEG-based linkers. PEG linkers may vary in length, for example, from 1-1000, from 1-500, from 1-11, from 1-100, from 1-50, or from 1-10 subunits. In some embodiments, a PEG linker comprises less than 100 subunits. In some embodiments, a PEGlinker comprises more than 100 subunits. In some embodiments, a PEG linker comprises more than 500 subunits. In some embodiments, a PEG linker comprises more than 1000 subunits. In some instances, a suitable PEG linker (or a branch thereof) may comprise at least 10 subunits, at least 20 subunits, at least 30 subunits, at least 40 subunits, at least 50 subunits, at least 60 subunits, at least 70 subunits, at least 80 subunits, at least 90 subunits, at least 100 subunits, at least 200 subunits, at least 300 subunits, at least 400 subunits, at least 500 subunits, at least 600 subunits, at least 700 subunits, at least 800 subunits, at least 900 subunits, or at least 1,000 subunits. In some instances, the PEG linker (or a branch thereof) comprises at most 1,000 subunits, at most 900 subunits, at most 800 subunits, at most 700 subunits, at most 600 subunits, at most 500 subunits, at most 400 subunits, at most 300 subunits, at most 200 subunits, at most 100 subunits, at most 90 subunits, at most 80 subunits, at most 70 subunits, at most 60 subunits, at most 50 subunits, at most 40 subunits, at most 30 subunits, at most 30 subunits, or at most 10 subunits. Any of the lower and upper values described in this paragraph may be combined to form a range included within the present disclosure, for example, in some instances a suitable PEG linker (or a branch thereof) may comprise from about 90 subunits to about 400 subunits.

In some embodiments, the linker (e.g., PEG linker) has an apparent average molecular weight, as measured by mass spectrometry, by electrophoretic methods, by size exclusion chromatography, by reverse-phase chromatography, or by any other means as known in the art for the estimation or measurement of the molecular weight of a polymer. In some instances, the apparent average molecular weight of the linker selected for conjugation may be less than about 1,000 Da, less than about 2,000 Da, less than about 3,000 Da, less than about 4,000 Da, less than about 5,000 Da, less than about 7,500 Da, less than about 10,000 Da, less than about 15,000 Da, less than about 20,000 Da, less than about 50,000 Da, less than about 100,000 Da, or less than about 200,000 Da. In some instances, the apparent average molecular weight of the linker selected for conjugation may be more than about 1,000 Da, more than about 2,000 Da, more than about 3,000 Da, more than about 4,000 Da, more than about 5,000 Da, more than about 7,500 Da, more than about 10,000 Da, more than about 15,000 Da, more than about 20,000 Da, more than about 50,000 Da, more than about 100,000 Da, or more than about 200,000 Da.

Examples of other suitable linkers may include, but are not limited to, poly-T and poly-A oligonucleotide strands (e.g., ranging from about 1 base to about 1,000 bases in length), peptide linkers (e.g., poly-glycine or poly-alanine ranging from about 1 residue to about 1,000 residues in length), or carbon-chain linkers (e.g., C6, C12, C18, C24, etc.).

In some embodiments, the linker contains an N-hydroxysuccinimide ester (NHS) group. In some embodiments, the linker contains a maleimide group. In some embodiments, the linker contains an NHS group and a maleimide group. The NHS group of a linker may then react with a primary amine on a nucleotide or other base-pairing moiety, thereby creating a covalent attachment without modifying or destroying the maleimide group. Such a functionalized nucleotide may then be covalently attached to the enzyme by reaction of the maleimide group with a cysteine residue of the enzyme.

Connection of the nucleotide can be achieved by the formation of a disulfide (forming a readily cleavable connection), formation of an amide, formation of an ester, protein-ligand linkage (e.g., biotin-streptavidin linkage), by alkylation (e.g., using a substituted iodoacetamide reagent) or forming adducts using aldehydes and amines or hydrazines.

In some embodiments, the linker contains, e.g., a maltose group, a biotin group, an 02-benzylcytosine group or 02-benzylcytosine derivative, an 06-benzylguanine group, or an 06-benzylguanine derivative. The NHS group of a linker may then react with a primary amine on a nucleotide, thereby creating a covalent attachment without modifying or destroying the maltose group, biotin group, 02-benzylcytosine group or 02-benzylcytosine derivative, 06-benzylguanine group, or 06-benzylguanine derivative. Such a functionalized nucleotide may then be covalently or non-covalently attached to the enzyme by reaction of the maltose group, biotin group, 02-benzylcytosine group or 02-benzylcytosine derivative, 06-benzylguanine group, or 06-benzylguanine derivative with a suitable functional group or binding partner attached to the enzyme.

Branched PEG molecules allow for simultaneous coupling of protein, dye(s), and nucleotide(s), such that multiple aspects of the compositions described herein may be present within a single reagent. Examples of suitable branched PEG molecules include, but are not limited to, PEG molecules comprising at least 4 branches, at least 8 branches, at least 16 branches, or at least 32 branches. Alternatively, it is contemplated that each individual element may be provided separately.

The length of the linker may vary depending on the type of nucleotide (or other base-pairing moiety) and the enzyme (or other nucleic acid binding moiety). In some instances, the enzyme linked nucleotide should have a length effective to allow the nucleotide or nucleotide analog to pair with a complementary nucleotide while precluding incorporation of the nucleotide or nucleotide analog into the 3′ end of a polynucleotide. In some instances, the linker length in the enzyme linked nucleotide is different for each different nucleotide or nucleotide analog. In some instances, the length of the linker will be defined as its persistence length, corresponding to the root-mean-square (RMS)distance between the ends of the linker as characterized by dynamic simulations, 2-D trapping experiments, or ab initio calculations. Such simulation, experiments, calculations can be based on statistical distributions of polymers in compact, collapsed, or fluid states as required by the solution, suspension, or fluid conditions present. In some instances, a linker may have persistence length from 0.1 to 1,000 nm, from 0.6 to 500 nm, for from 0.6 to 400 nm. In some instances, a linker may have a persistence length of 0.6, 3.1, 12.7, 22.3, 31.8, 47.7, 95.5, 190.9, 381.8, 763.8 nm, or 989.5 nm or a range defined by or comprising any two or more of these values. In some instances, a linker may have a persistence length of at least 0.1, at least 0.2, at least 0.4, at least 1, at least 2, at least 4, at least 10, at least 20, at least 30, at least 40, at least 50, at least 60, at least 80, at least 90, at least 100, at least 200, at least 300, at least 400, at least 500, at least 700, or at least 1,000 nm, or a persistence length in a range defined by or comprising any two or more of these values. In some instances, linkers provided for one nucleotide may be longer or shorter than the linker provided for another nucleotide. For example, in some instances, dTTP may be linked to a nucleic acid binding moiety thought a longer linker than is used to tether dGTP, or vice versa.

In some instances, a linker for connecting the nucleotide to the enzyme can have a persistence length of about 0.1-1,000 nm, 0.5-500 nm, 0.5-400 nm, 0.5-300 nm, 0.5-200 nm, 0.5-100 nm, 0.5-50 nm, 0.6-500 nm, 0.6-400 nm, 0.6-300 nm, 0.6-200 nm, 0.6-100 nm, 0.6-50 nm, 1-500 nm, 1-400 nm, 1-300 nm, 1-200 nm, 1-100 nm, 1.5-500 nm, 1.5-400 nm, 1.5-300 nm, 1.5-200 nm, 1.5-100 nm, 1.5-50 nm, 1-50 nm, 5-500 nm, 5-400 nm, 5-300 nm, 5-200 nm, 5-100 nm, or 5-50 nm. In some instances, a linker may have a persistence length of about 0.1, 0.5, 0.6, 1.0, 1.5, 1.8, 2.0, 2.5, 3.0, 3.1, 4.0, 5.0, 6.0, 7.0, 8.0, 9.0, 10.0, 12.7, 22.3, 31.8, 47.7, 95.5, 190.9, or 381.8 nm, or a persistence length in a range defined by or comprising any two or more of these values. In some instances, a linker may have a persistence length of greater than about 0.1, 0.5, 0.6, 1.0, 1.5, 1.8, 2.0, 2.5, 3.0, 3.1, 4.0, 5.0, 6.0, 7.0, 8.0, 9.0, 10.0, 12.7, 22.3, 31.8, 47.7, 95.5, 190.9, or 381.8 nm. In some instances, the linker may have a persistence length of shorter than about 5, 10, 20, 30, 40, 50, 60, 80, 100, 200, 300, 400, 500, 700, or 1,000 nm. In some instances, a linker may have a persistence length of 0.1, 0.2, 0.4, 1, 2, 4, 10, 20, 30, 40, 50, 60, 80, 100, 200, 300, 400, 500, 700, or 1,000 nm, or a persistence length in a range defined by or comprising any two or more of these values.

The polymerase molecules of the disclosure can be site-specifically conjugated to a terminal phosphate group of a nucleoside to form a tethered molecule via a chemical linker. In some embodiments, the chemical linker is an acid-labile linker. In some embodiments, the chemical linker is a base-labile linker. In some embodiments, the chemical linker can be cleaved with irradiation. In some embodiments, the chemical linker can be cleaved with an enzyme, for example, a peptidase, or esterase. In some embodiments, the chemical linker is a pH-sensitive linker. In some embodiments, the chemical linker is an amine-to-thiol crosslinker, such as PEG4-SPDP. In some embodiments, the chemical linker is a thiomaleamic acid linker. In some embodiments, the chemical linker is a silane. In some embodiments, the chemical linker is cleavable using pH or fluoride.

The polymerase chemically linked to the nucleotide can be cleaved using a chemical reagent. In some embodiments, the chemical linker is a disulfide bond, which can be cleaved by a reducing agent. In some embodiments, a disulfide chemical linker is cleaved using B-mercaptoethanol (BME). In some embodiments, the chemical linker is a base-cleavable bond, such as an ester (e.g., succinate). In some embodiments, the chemical linker is a base-cleavable linker that can be cleaved using ammonia or trimethylamine. In some embodiments, the chemical linker is a quaternary ammonium salt that can be cleaved using diisopropylamine. In some embodiments, the chemical linker is a urethane that can be cleaved by a base, such as aqueous sodium hydroxide.

In some embodiments, the chemical linker is an acid-cleavable linker. In some embodiments, the chemical linker is a benzyl alcohol derivative. In some embodiments, the acid-cleavable linker can be cleaved using trifluoroacetic acid. In some embodiments, the chemical linker teicoplanin aglycone, which can be cleaved by treatment with trifluoroacetic acid and a base. In some embodiments, the chemical linker is an acetal or thioacetal, which can be cleaved by trifluoroacetic acid. In some embodiments, the chemical linker is a thioether that can be cleaved by hydrogen fluoride or cresol. In some embodiments, the chemical linker is a sulfonyl group that can be cleaved by trifluoromethane sulfonic acid, trifluoroacetic acid, or thioanisole. In some embodiments, the chemical linker comprises a nucleophile-cleavable site, such as a phthalimide that can be cleaved by treatment with a hydrazine. In some embodiments, the chemical linker can be an ester that can be cleaved with aluminum trichloride.

In some embodiments, the chemical linker is a Weinreb amide, which can be cleaved by lithium aluminum hydride). In some embodiments, the chemical linker is a phosphorothionate that can be cleaved by silver or mercury ions. In some embodiments, the chemical linker can be a diisopropyldialkoxysilyl group that can be cleaved by fluoride ions. In some embodiments, the chemical linker can be a diol that can be cleaved by sodium periodate. In some embodiments, the chemical linker can be an azobenzene that can be cleaved by sodium dithionate.

In some embodiments, the chemical linker is a photo-cleavable linker. In some embodiments, the photo-cleavable linker is an orthonitrobenzyl-based linker, phenacyl linker, alkoxybenzoin linker, chromium arene complex linker, NpSSMpact linker, or pivaloylglycol linker. In some embodiments, the photo-cleavable linker can be cleaved by irradiating the linker at about 300 to 500 nm. In some embodiments, the photo-cleavable linker can be cleaved by irradiating the linker at about 300 to 400, 300 to 450, 300 to 500, 350 to 370, 350 to 400, 350 to 450, 350 to 500, 400 to 420, 400 to 450, or 400 to 500 nm. In some embodiments, the photo-cleavable linker can be cleaved by irradiating the linker at about 312 nm. In some embodiments, the photo-cleavable linker can be cleaved by irradiating the linker at about 365 nm. In some embodiments, the photo-cleavable linker can be cleaved by irradiating the linker at about 405 nm. In some embodiments, the photo-cleavable linker is irradiated for about 1, 2, 3, 4, 5, 6, 7, 8, 9, or 10 minutes. In some embodiments, the photo-cleavable linker is irradiated for at least about 1, 2, 3, 4, 5, 6, 7, 8, 9, or 10 minutes. In some embodiments, the photo-cleavable linker is irradiated for at most about 1, 2, 3, 4, 5, 6, 7, 8, 9, or 10 minutes. In some embodiments, the photo-cleavable linker is irradiated for about 1-3, 1-5, 1-8, 1-10, 2-4, 2-6, 2-8, 2-10, 3-5, 3-7, 3-9, 3-10, 4-6, 4-8, 4-10, 5-8, 5-10, 6-8, 6-10, 7-9, 7-10, 8-10, or 9-10 minutes.

In some embodiments, the chemical linker is selected from the group consisting of a silyl linker, an alkyl linker, a polyether linker, a polysulfonyl linker, a polysulfoxide linker, and any combination thereof.

In some embodiments, the linker is cleaved by an enzyme. In some embodiments, the enzyme is a protease, an esterase, a glycosylase, or a peptidase. In some embodiments, the cleaving enzyme breaks bonds in the polymerase. In some embodiments, the cleaving enzyme directly cleaves the linked nucleoside.

Provided herein are methods for enzymatically synthesizing polynucleotides comprising using various buffers. The buffers, in some embodiments, are used in a coupling reaction, deblocking reaction, washing solution, or combinations thereof. In some embodiments, the buffer comprises sodium cacodylate, Tris-HCl, MgCl₂, ZnSO₄, sodium acetate, or combinations thereof.

The enzymatic methods described herein can be used to synthesize biopolymers. Biopolymers include, but are not limited to, polynucleotides or oligonucleotides. Polynucleotide sequences described herein may be, unless stated otherwise, comprise DNA or RNA. In some cases, the polynucleotide comprises RNA. In some instances, RNA comprises short interfering RNA (siRNA), short hairpin RNA (shRNA), microRNA (miRNA), double-stranded RNA (dsRNA), transfer RNA (tRNA), ribosomal RNA (rRNA), or heterogeneous nuclear RNA (hnRNA). In some instances, RNA comprises shRNA. In some instances, RNA comprises miRNA. In some instances, RNA comprises dsRNA. In some instances, RNA comprises tRNA. In some instances, RNA comprises rRNA. In some instances, RNA comprises hnRNA. In some instances, the polynucleotide is a phosphorodiamidate morpholino oligomers (PMO), which are short single-stranded polynucleotide analogs that are built upon a backbone of morpholine rings connected by phosphorodiamidate linkages. In some instances, the RNA comprises siRNA. In some instances, the polynucleotide comprises siRNA.

In some embodiments, the polynucleotide is from about 8 to about 50 nucleotides in length. In some embodiments, the polynucleotide is from about 10 to about 50 nucleotides in length. In some instances, the polynucleotide is about 10, 15, 18, 20, 22, 25, 30, 35, 40, 45, or 50 nucleotides in length. In some instances, the polynucleotide is from about 10 to about 30, from about 15 to about 30, from about 18 to about 25, form about 18 to about 24, from about 19 to about 23, or from about 20 to about 22 nucleotides in length.

In some embodiments, the polynucleotide is about 50 nucleotides in length. In some instances, the polynucleotide is about 45 nucleotides in length. In some instances, the polynucleotide is about 40 nucleotides in length. In some instances, the polynucleotide is about 35 nucleotides in length. In some instances, the polynucleotide is about 30 nucleotides in length. In some instances, the polynucleotide is about 25 nucleotides in length. In some instances, the polynucleotide is about 20 nucleotides in length. In some instances, the polynucleotide is about 19 nucleotides in length. In some instances, the polynucleotide is about 18 nucleotides in length. In some instances, the polynucleotide is about 17 nucleotides in length. In some instances, the polynucleotide is about 16 nucleotides in length. In some instances, the polynucleotide is about 15 nucleotides in length. In some instances, the polynucleotide is about 14 nucleotides in length. In some instances, the polynucleotide is about 13 nucleotides in length. In some instances, the polynucleotide is about 12 nucleotides in length. In some instances, the polynucleotide is about 11 nucleotides in length. In some instances, the polynucleotide is about 10 nucleotides in length. In some instances, the polynucleotide is about 8 nucleotides in length. In some instances, the polynucleotide is between about 8 and about 50 nucleotides in length. In some instances, the polynucleotide is between about 10 and about 50 nucleotides in length. In some instances, the polynucleotide is between about 10 and about 45 nucleotides in length. In some instances, the polynucleotide is between about 10 and about 40 nucleotides in length. In some instances, the polynucleotide is between about 10 and about 35 nucleotides in length. In some instances, the polynucleotide is between about 10 and about 30 nucleotides in length. In some instances, the polynucleotide is between about 10 and about 25 nucleotides in length. In some instances, the polynucleotide is between about 10 and about 20 nucleotides in length. In some instances, the polynucleotide is between about 15 and about 25 nucleotides in length. In some instances, the polynucleotide is between about 15 and about 30 nucleotides in length. In some instances, the polynucleotide is between about 12 and about 30 nucleotides in length.

In some embodiments, the DNA or RNA is chemically modified. In some embodiments, the polynucleotide comprises natural or synthetic or artificial nucleotide analogues or bases. In some cases, the polynucleotide comprises combinations of DNA, RNA and/or nucleotide analogues. The polynucleotides may be modified using LNA monomers. In some embodiments, the polynucleotides are modified using MOE, ANA, FANA, PS, or combinations thereof.

In some instances, the synthetic or artificial nucleotide analogues or bases comprise modifications at one or more of ribose moiety, phosphate moiety, nucleoside moiety, or a combination thereof. In some embodiments, nucleotide analogues or artificial nucleotide base comprise a nucleic acid with a modification at a 2′ hydroxyl group of the ribose moiety. In some instances, the modification includes an H, OR, R, halo, SH, SR, NH₂, NHR, NR₂, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, and disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

Modified polynucleotides may also contain one or more substituted sugar moieties. In some embodiments, the modified polynucleotide comprises one of the following at the 2′ position: OH; F; O—, S—, or N-alkyl; O—, S—, or N-alkenyl; O—, S- or N-alkynyl; or O alkyl-O-alkyl, wherein the alkyl, alkenyl and alkynyl may be substituted or unsubstituted C to CO alkyl or C₂ to C₁₀ alkenyl and alkynyl. Particularly preferred are O(CH₂)ₙOₘCH₃, O(CH₂)ₙ, OCH₃, O(CH₂), NH₂, O(CH₂), CH₃, O(CH₂)ONH₂, and O(CH₂)ₙON (CH₃)₂ where n and m can be from 1 to about 10. In some embodiments, the modified polynucleotide comprises one of the following at the 2′ position: C to CO, (lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a polynucleotide, or a group for improving the pharmacodynamic properties of a polynucleotide, and other substituents having similar properties. In some embodiments, modification comprises 2′-methoxyethoxy (2′-O—CH₂CH₂OCH₃, also known as 2′-O-(2-methoxyethyl) or 2′-MOE) i.e., an alkoxyalkoxy group. A further preferred modification comprises 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

In some embodiments, the polynucleotide one or more of the artificial nucleotide analogues described herein. In some instances, the polynucleotide comprises 1, 2, 3, 4, 5, 6, 7, 8, 9, 10, 11, 12, 13, 14, 15, 16, 17, 18, 20, 25, or more of the artificial nucleotide analogues described herein. In some embodiments, the artificial nucleotide analogues include 2′-O-methyl, 2′-O-methoxyethyl(2′-O-MOE), 2′-O-aminopropyl, 2′-deoxy, T-deoxy-2′-fluoro, 2′-O-aminopropyl(2′-O-AP), 2′-O-dimethylaminoethyl(2′-O-DMAOE), 2′-O-dimethylaminopropyl(2′-O-DMAP), T-O-dimethylaminoethyloxyethyl(2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N₃—P5′-phosphoramidites, or a combination thereof. In some instances, the polynucleotide comprises 1, 2, 3, 4, 5, 6, 7, 8, 9, 10, 11, 12, 13, 14, 15, 16, 17, 18, 20, 25, or more of the artificial nucleotide analogues selected from 2′-O-methyl, 2′-O-methoxyethyl(2′-O-MOE), 2′-O-aminopropyl, 2′-deoxy, T-deoxy-2′-fluoro, 2′-O-aminopropyl(2′-O-AP), 2′-O-dimethylaminoethyl(2′-O-DMAOE), 2′-O-dimethylaminopropyl(2′-O-DMAP), T-O-dimethylaminoethyloxyethyl(2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the polynucleotide comprises 1, 2, 3, 4, 5, 6, 7, 8, 9, 10, 11, 12, 13, 14, 15, 16, 17, 18, 20, 25, or more of 2′-O-methyl modified nucleotides. In some instances, the polynucleotide comprises 1, 2, 3, 4, 5, 6, 7, 8, 9, 10, 11, 12, 13, 14, 15, 16, 17, 18, 20, 25, or more of 2′-O-methoxyethyl(2′-O-MOE) modified nucleotides. In some instances, the polynucleotide comprises 1, 2, 3, 4, 5, 6, 7, 8, 9, 10, 11, 12, 13, 14, 15, 16, 17, 18, 20, 25, or more of thiolphosphonate nucleotides.

In some embodiments, the modifications comprise 2′-methoxy (2′-OCH₃), 2′-aminopropoxy (2′-OCH₂CH₂CH₂NH₂) and 2′-fluoro (2′-F). Similar modifications may also be made at other positions on the polynucleotide, particularly 3′ position of the sugar on 3′ terminal nucleotide or in 2′-5′ linked polynucleotides and 5′ position of 5′ terminal nucleotide. In some embodiments, the polynucleotide comprises sugar mimetics such as cyclobutyl moieties in place of the pentofuranosyl sugar.

Polynucleotides may also comprise nucleobase (“base”) modifications or substitutions. As used herein, “unmodified” or “natural” nucleotides comprise the purine bases adenine (A) and guanine (G), and the pyrimidine bases thymine (T), cytosine (C) and uracil (U). Modified nucleotides comprise other synthetic and natural nucleotides such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

In some embodiments, the polynucleotide backbone is modified. In some embodiments, the polynucleotide backbone comprises, but not limited to, phosphorothioates, chiral phosphorothioates, phosphorodithioates, phosphotriesters, aminoalkylphosphotriesters, methyl and other alkyl phosphonates comprising 3′ alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

In some embodiments, the modified polynucleotide backbone does not comprise a phosphorus atom therein and comprise backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In some embodiments, the polynucleotide is modified by chemically linking the polynucleotide to one or more moieties or conjugates. Exemplary moieties include, but are not limited to, lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or Adamantane acetic acid, a palmityl moiety, or an octadecylamine or hexylamino-carbonyl-t oxycholesterol moiety.

When a non-naturally occurring chemical linker is cleaved from a polynucleotide or polynucleotide, the remaining chemical moiety is referred to as a “scar.” In some embodiments, the scar is an olefin or alkyne moiety. The methods as described herein, in some embodiments, do not leave a scar. In some embodiments, no scar remains after the linked phosphate is cleaved.

The method of enzymatic polynucleotide synthesis disclosed herein can have a coupling efficiency of at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9%. In some embodiments, the method can have a coupling efficiency of at least 99.5%. In some embodiments, the method can have a coupling efficiency of at least 99.7%. In some embodiments, the method can have a coupling efficiency of at least 99.9%.

The method of enzymatic polynucleotide synthesis disclosed herein can have a coupling efficiency of about 95%, about 95.5%, about 96%, about 96.5%, about 97%, about 97.5%, about 98%, about 98.5%, about 99%, about 99.5%, about 99.6%, about 99.7%, about 99.8%, or about 99.9%. In some embodiments, the method can have a coupling efficiency of about 99.5%. In some embodiments, the method can have a coupling efficiency of about 99.7%. In some embodiments, the method can have a coupling efficiency of about 99.9%.

The method of enzymatic polynucleotide synthesis described herein can have a total average error rate of less than about 1 in 100, less than about 1 in 200, less than about 1 in 300, less than about 1 in 400, less than about 1 in 500, less than about 1 in 1000, less than about 1 in 2000, less than about 1 in 5000, less than about 1 in 10000, less than about 1 in 15000, or less than about 1 in 20000 bases. In some embodiments, the total average error rate is less than about 1 in 100. In some embodiments, the total average error rate is less than about 1 in 200. In some embodiments, the total average error rate is less than about 1 in 500. In some embodiments, the total average error rate is less than about 1 in 1000.

The method of enzymatic polynucleotide synthesis described herein can have a total average error rate of less than about 95%, less than about 96%, less than about 97%, less than about 98%, less than about 99%, less than about 99.5%, less than about 99.6%, less than about 99.7%, less than about 99.8%, or less than about 99.9%. In some embodiments, the method can have a total average error rate of less than about 99.5%. In some embodiments, the method can have a total average error rate of less than about 99.7%. In some embodiments, the method can have a total average error rate of less than about 99.9%.

The error rates of the method disclosed herein are for at least 50%, 60%, 70%, 80%, 90%, 95%, 98%, 99%, 99.5%, or more of the polynucleotides synthesized. In some embodiments, the error rates are for at least 60% of the synthesized polynucleotides. In some embodiments, the error rates are for at least 80% of the synthesized polynucleotides. In some embodiments, the error rates are for at least 90% of the synthesized polynucleotides. In some embodiments, the error rates are for at least 99% of the synthesized polynucleotides. Individual types of error rates include mismatches, deletions, insertions, and/or substitutions for the polynucleotides synthesized on the substrate. The term “error rate” refers to a comparison of the collective amount of synthesized biopolymer to an aggregate of predetermined biopolymer sequence.

The method of enzymatic polynucleotide synthesis disclosed herein can extend a primer by a single nucleotide in from about 1 second (sec) to about 20 sec. In some embodiments, the method can extend a single nucleotide in from about 1 sec to about 5 sec. In some embodiments, the method can extend a single nucleotide in from about 5 sec to about 10 sec. In some embodiments, the method can extend a single nucleotide in from about 10 sec to about 15 sec. In some embodiments, the method can extend a single nucleotide in from about 15 sec to about 20 sec. In some embodiments, the method can extend a single nucleotide in from about 10 sec to about 20 sec.

The method of enzymatic polynucleotide synthesis disclosed herein can extend a primer by a single nucleotide in about 1 second (sec), about 2 sec, about 3 sec, about 4 sec, about 5 sec, about 6 sec, about 7 sec, about 8 sec, about 9 sec, about 10 sec, about 11 sec, about 12 sec, about 13 sec, about 14 sec, about 15 sec, about 16 sec, about 17 sec, about 18 sec, about 19 sec, or about 20 sec. In some embodiments, the method can extend a single nucleotide in about 5 sec. In some embodiments, the method can extend a single nucleotide in about 10 sec. In some embodiments, the method can extend a single nucleotide in about 15 sec. In some embodiments, the method can extend a single nucleotide in about 20 sec.

The method of enzymatic polynucleotide synthesis disclosed herein can extend a polynucleotide by at least about 10 nucleotides per hour. In some instances, the method extends a polynucleotide by at least about 5, 10, 15, 20, 25, 30, 35, 40, 45, 50, or more than 50 nucleotides per hour.

The synthesized polynucleotides of the disclosure can be between about 50 bases to about 1000 bases. In some embodiments, the synthesized polynucleotides comprise at least 10, at least 20, at least 30, at least 40, at least 50, at least 60, at least 70, at least 80, at least 90, at least 100, at least 125, at least 150, at least 175, at least 200, at least 225, at least 250, at least 275, at least 300, at least 325, at least 350, at least 375, at least 400, at least 425, at least 450, at least 475, at least 500, at least 600, at least 700, at least 800, at least 900, at least 1000, at least 1100, at least 1200, at least 1300, at least 1400, at least 1500, at least 1600, at least 1700, at least 1800, at least 1900, or at least 2000 bases. In some embodiments, the synthesized polynucleotides comprise about 10, about 20, about 30, about 40, about 50, about 60, about 70, about 80, about 90, about 100, about 125, about 150, about 175, about 200, about 225, about 250, about 275, about 300, about 325, about 350, about 375, about 400, about 425, about 450, about 475, about 500, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, 4000, 5000, or more than 5000 bases.

In some embodiments, the polymerase-nucleotide conjugates can comprise additional moieties that terminate elongation of a nucleic acid once the tethered nucleic acid is incorporated. In some embodiments, a 3′ O-modified or base-modified reversible terminator deoxynucleoside triphosphate (RTdNTP) is tethered to the polymerase. In some embodiments, the reversible terminator may be coupled to the oxygen atom of the 3-prime hydroxyl group of the nucleotide pentose (e.g., 3′-O-blocked reversible terminator). Alternatively, or in addition to, the reversible terminator may be coupled to the nucleobase of the nucleotide (e.g., 3′-unblocked reversible terminator). In some embodiments, a reversible terminator nucleotide is a chemically modified nucleoside triphosphate analog that stops elongation once incorporated into the nucleic acid molecule. When a conjugate comprising a polymerase and an RTdNTP is used for the extension of nucleic acids, cleavage of the linker and deprotection of the RTdNTP may be required to enable an extended nucleic acid to undergo further nucleotide addition. The reversible terminator may include a detectable label. The reversible terminator may comprise an allyl, hydroxylamine, acetate, benzoate, phosphate, azidomethyl, or amide group. The reversible terminator may be removed by treatment with a reducing agent, acid or base, organic solvents, ionic surfactants, photons (photolysis), or any combination thereof.

In a conjugate, the linker is considered to be at least the atoms that connect the α-phosphate of a nucleotide to a C_{α} atom in the backbone of the polymerase. In some embodiments, the polymerase and the nucleotide are covalently linked, and the distance between the linked atom of the nucleotide and the C_{α} atom in the backbone of the polymerase is from about 4 Å to about 100 Å. In some embodiments, the distance between the linked atom of the nucleoside and the C_{α} atom in the backbone of the polymerase is about 5 Å to about 20 Å. In some embodiments, the distance between the linked atom of the nucleoside and the C_{α} atom in the backbone of the polymerase is about 20 Å to about 50 Å. In some embodiments, the distance between the linked atom of the nucleoside and the C_{α} atom in the backbone of the polymerase is about 50 Å to about 75 Å. In some embodiments, the distance between the linked atom of the nucleoside and the C_{α} atom in the backbone of the polymerase is about 75 Å to about 100 Å.

In some embodiments, the linker is joined to the base of the nucleotide at an atom that is not involved in base pairing. In some embodiments, the linker is at least the atoms that connect a Cq atom in the backbone of the polymerase to a terminal phosphate group of the nucleotide.

The linker should be sufficiently long to allow the nucleoside triphosphate to access the active site of the polymerase to which it is tethered. The polymerase of a conjugate can catalyze the addition of the nucleotide to which it is linked onto the 3′ end of a nucleic acid.

### Methods of Use

The compositions and methods described herein can be used in nucleic acid assembly. In some embodiments, the nucleic acid is a DNA. In some embodiments, the nucleic acid is an RNA. In some embodiments, the compositions and methods described herein can be used to assemble nucleic acids that are about 8 to about 100 nucleotides in length. In some embodiments, the compositions and methods described herein can be used to assemble nucleic acids that are about 8 to about 50 nucleotides in length. In some embodiments, the compositions and methods described herein can be used to assemble nucleic acids that are about 50 nucleic acids in length.

The compositions and methods described herein can be used in place of Gibson assembly. The compositions and methods described herein can be used to join multiple DNA fragments in a single, isothermal reaction. In some embodiments, the compositions and methods described herein can be used to combine 1, 2, 3, 4, 5, 6, 7, 8, 9, 10, 11, 12, 13, 14, 15, 16, 17, 18 19, or 20 DNA fragments based on sequence identity. In some embodiments, the compositions or methods described herein can be used to combine 10 DNA fragments. In some embodiments, the compositions or methods described herein can be used to combine 15 DNA fragments. In some embodiments, the compositions or methods described herein can be used to combine 20 DNA fragments. In some embodiments, the DNA fragments to be combined contain an about 15, about 20, about 25, about 30, about 35, about 40, about 45, or about 50 base pair overlap with adjacent DNA fragments. In some embodiments, the DNA fragments to be combined using the methods described herein contain an about 20 base pair overlap with adjacent DNA fragments. In some embodiments, the DNA fragments to be combined using the methods described herein contain an about 30 base pair overlap with adjacent DNA fragments. In some embodiments, the DNA fragments to be combined using the methods described herein contain an about 40 base pair overlap with adjacent DNA fragments.

Described herein are compositions and methods for gene assembly to generate a gene library. The gene library can comprise a collection of genes. In some embodiments, the collection comprises at least 100 different preselected synthetic genes that can be of at least 0.5 kb length with an error rate of less than 1 in 3000 bp compared to predetermined sequences comprising the genes. The collection may comprise at least 100 different preselected synthetic genes that can be each of at least 0.5 kb length. At least 90% of the preselected synthetic genes may comprise an error rate of less than 1 in 3000 bp compared to predetermined sequences comprising the genes. Desired predetermined sequences may be supplied by any method, typically by a user, e.g. a user entering data using a computerized system. In various embodiments, synthesized nucleic acids are compared against these predetermined sequences, in some cases by sequencing at least a portion of the synthesized nucleic acids, e.g. using next-generation sequencing methods. In some embodiments related to any of the gene libraries described herein, at least 90% of the preselected synthetic genes comprise an error rate of less than 1 in 5000 bp compared to predetermined sequences comprising the genes. In some embodiments, at least 0.05% of the preselected genes are error free. In some embodiments, at least 0.5% of the preselected genes are error free. In some embodiments, at least 90% of the preselected genes comprise an error rate of less than 1 in 3000 bp compared to predetermined sequences comprising the genes. In some embodiments, at least 90% of the preselected genes are error free or substantially error free. In some embodiments, the preselected genes comprise a deletion rate of less than 1 in 3000 bp compared to predetermined sequences comprising the genes. In some embodiments, the preselected genes comprise an insertion rate of less than 1 in 3000 bp compared to predetermined sequences comprising the genes. In some embodiments, the preselected genes comprise a substitution rate of less than 1 in 3000 bp compared to predetermined sequences comprising the genes. In some embodiments, the gene library as described herein further comprises at least 10 copies of each gene. In some embodiments, the gene library as described herein further comprises at least 100 copies of each gene. In some embodiments, the gene library as described herein further comprises at least 1000 copies of each gene. In some embodiments, the gene library as described herein further comprises at least 1000000 copies of each gene. In some embodiments, the collection of genes as described herein comprises at least 500 genes. In some embodiments, the collection comprises at least 5000 genes. In some embodiments, the collection comprises at least 10000 genes. In some embodiments, the preselected genes are at least 1 kb. In some embodiments, the preselected genes are at least 2 kb. In some embodiments, the preselected genes are at least 3 kb. In some embodiments, the predetermined sequences comprise less than 20 bp in addition compared to the preselected genes. In some embodiments, the predetermined sequences comprise less than 15 bp in addition compared to the preselected genes. In some embodiments, at least one of the genes differs from any other gene by at least 0.1%. In some embodiments, each of the genes differs from any other gene by at least 0.1%. In some embodiments, at least one of the genes differs from any other gene by at least 10%. In some embodiments, each of the genes differs from any other gene by at least 10%. In some embodiments, at least one of the genes differs from any other gene by at least 2 base pairs. In some embodiments, each of the genes differs from any other gene by at least 2 base pairs. In some embodiments, the gene library as described herein further comprises genes that are of less than 2 kb with an error rate of less than 1 in 20000 bp compared to preselected sequences of the genes. In some embodiments, a subset of the deliverable genes is covalently linked together. In some embodiments, a first subset of the collection of genes encodes for components of a first metabolic pathway with one or more metabolic end products. In some embodiments, the gene library as described herein further comprises selecting of the one or more metabolic end products, thereby constructing the collection of genes. In some embodiments, the one or more metabolic end products comprise a biofuel. In some embodiments, a second subset of the collection of genes encodes for components of a second metabolic pathway with one or more metabolic end products. In some embodiments, the gene library is in a space that is less than 100 m³. In some embodiments, the gene library is in a space that is less than 1 m³.

In some instances, described herein are methods of constructing a gene library. The method may comprise the steps of: entering before a first timepoint, in a computer readable non-transient medium at least a first list of genes and a second list of genes, wherein the genes are at least 500 bp and when compiled into a joint list, the joint list comprises at least 100 genes; synthesizing more than 90% of the genes in the joint list before a second timepoint, thereby constructing a gene library with deliverable genes. In some embodiments, the second timepoint is less than a month apart from the first timepoint.

In practicing any of the methods of constructing a gene library as provided herein, the method as described herein further comprises delivering at least one gene at a second timepoint. In some embodiments, at least one of the genes differs from any other gene by at least 0.1% in the gene library. In some embodiments, each of the genes differs from any other gene by at least 0.1% in the gene library. In some embodiments, at least one of the genes differs from any other gene by at least 10% in the gene library. In some embodiments, each of the genes differs from any other gene by at least 10% in the gene library. In some embodiments, at least one of the genes differs from any other gene by at least 2 base pairs in the gene library. In some embodiments, each of the genes differs from any other gene by at least 2 base pairs in the gene library. In some embodiments, at least 90% of the deliverable genes are error free. In some embodiments, the deliverable genes comprises an error rate of less than 1/3000 resulting in the generation of a sequence that deviates from the sequence of a gene in the joint list of genes. In some embodiments, at least 90% of the deliverable genes comprise an error rate of less than 1 in 3000 bp resulting in the generation of a sequence that deviates from the sequence of a gene in the joint list of genes. In some embodiments, genes in a subset of the deliverable genes are covalently linked together. In some embodiments, a first subset of the joint list of genes encode for components of a first metabolic pathway with one or more metabolic end products. In some embodiments, any of the methods of constructing a gene library as described herein further comprises selecting of the one or more metabolic end products, thereby constructing the first, the second or the joint list of genes. In some embodiments, the one or more metabolic end products comprise a biofuel. In some embodiments, a second subset of the joint list of genes encode for components of a second metabolic pathway with one or more metabolic end products. In some embodiments, the joint list of genes comprises at least 500 genes. In some embodiments, the joint list of genes comprises at least 5000 genes. In some embodiments, the joint list of genes comprises at least 10000 genes. In some embodiments, the genes can be at least 1 kb. In some embodiments, the genes are at least 2 kb. In some embodiments, the genes are at least 3 kb. In some embodiments, the second timepoint is less than 25 days apart from the first timepoint. In some embodiments, the second timepoint is less than 5 days apart from the first timepoint. In some embodiments, the second timepoint is less than 2 days apart from the first timepoint. It is noted that any of the embodiments described herein can be combined with any of the methods, devices or systems provided in the current disclosure.

In another aspect, a method of constructing a gene library is provided herein. The method comprises the steps of: entering at a first timepoint, in a computer readable non-transient medium a list of genes; synthesizing more than 90% of the list of genes, thereby constructing a gene library with deliverable genes; and delivering the deliverable genes at a second timepoint. In some embodiments, the list comprises at least 100 genes and the genes can be at least 500 bp. In still yet some embodiments, the second timepoint is less than a month apart from the first timepoint.

In practicing any of the methods of constructing a gene library as provided herein, in some embodiments, the method as described herein further comprises delivering at least one gene at a second timepoint. In some embodiments, at least one of the genes differs from any other gene by at least 0.1% in the gene library. In some embodiments, each of the genes differs from any other gene by at least 0.1% in the gene library. In some embodiments, at least one of the genes differs from any other gene by at least 10% in the gene library. In some embodiments, each of the genes differs from any other gene by at least 10% in the gene library. In some embodiments, at least one of the genes differs from any other gene by at least 2 base pairs in the gene library. In some embodiments, each of the genes differs from any other gene by at least 2 base pairs in the gene library. In some embodiments, at least 90% of the deliverable genes are error free. In some embodiments, the deliverable genes comprises an error rate of less than 1/3000 resulting in the generation of a sequence that deviates from the sequence of a gene in the list of genes. In some embodiments, at least 90% of the deliverable genes comprise an error rate of less than 1 in 3000 bp resulting in the generation of a sequence that deviates from the sequence of a gene in the list of genes. In some embodiments, genes in a subset of the deliverable genes are covalently linked together. In some embodiments, a first subset of the list of genes encode for components of a first metabolic pathway with one or more metabolic end products. In some embodiments, the method of constructing a gene library further comprises selecting of the one or more metabolic end products, thereby constructing the list of genes. In some embodiments, the one or more metabolic end products comprise a biofuel. In some embodiments, a second subset of the list of genes encode for components of a second metabolic pathway with one or more metabolic end products. It is noted that any of the embodiments described herein can be combined with any of the methods, devices or systems provided in the present disclosure.

In practicing any of the methods of constructing a gene library as provided herein, in some embodiments, the list of genes comprises at least 500 genes. In some embodiments, the list comprises at least 5000 genes. In some embodiments, the list comprises at least 10000 genes. In some embodiments, the genes are at least 1 kb. In some embodiments, the genes are at least 2 kb. In some embodiments, the genes are at least 3 kb. In some embodiments, the second timepoint as described in the methods of constructing a gene library is less than 25 days apart from the first timepoint. In some embodiments, the second timepoint is less than 5 days apart from the first timepoint. In some embodiments, the second timepoint is less than 2 days apart from the first timepoint. It is noted that any of the embodiments described herein can be combined with any of the methods, devices or systems provided in the present disclosure.

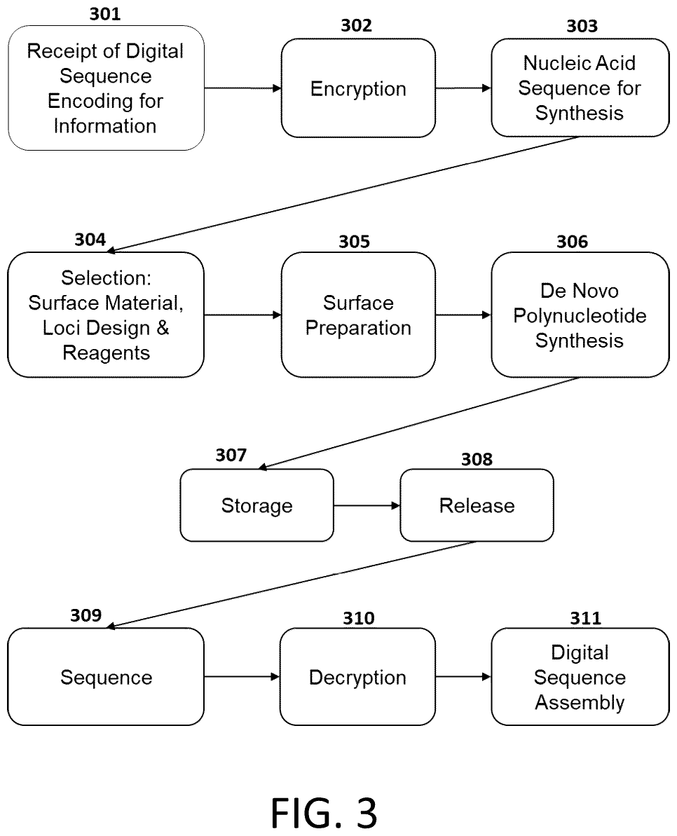

The compositions and methods descried herein can be used for DNA digital data storage. In some embodiments, the compositions and methods disclosed herein can be used to prepare DNA molecules for four bit information coding. An exemplary workflow is provided in FIG. 3. In a first step, a digital sequence encoding an item of information (i.e., digital information in a binary code for processing by a computer) is received 301. An encryption 302 scheme is applied to convert the digital sequence from a binary code to a nucleic acid sequence 303. A surface material for nucleic acid extension, a design for loci for nucleic acid extension (aka, arrangement spots), and reagents for nucleic acid synthesis are selected 304. The surface of a structure is prepared for nucleic acid synthesis 305. De novo polynucleotide synthesis is performed 306. The polynucleotides may be about 8 to 300 bases in length. In some instances, the polynucleotides are about 8, 10, 50, 80, 100, 110, 120, 130, 140, 150, 160, 170, 180, 190, 200, 250, or 300 bases in length. In some instances, the polynucleotides are at most about 8, 10, 50, 80, 100, 110, 120, 130, 140, 150, 160, 170, 180, 190, 200, 250, or 300 bases in length. In some instances, the polynucleotides are at least about 8, 10, 50, 80, 100, 110, 120, 130, 140, 150, 160, 170, 180, 190, 200, 250, or 300 bases in length. In some instances, the polynucleotides are about 10 to 100, 10 to 150, 10 to 200, 50 to 100, 50 to 150, 50 to 200, 100 to 150, 100 to 200, 100 to 300, 150 to 200, 150 to 250, 150 to 300, or 200 to 300 bases in length. The synthesized polynucleotides are stored 307 and available for subsequent release 308, in whole or in part. For example, select polynucleotides may be independently cleaved and released from the surface. In some instances, the polynucleotides are stored on the surface that they were synthesized on. However, in alternative instances, the polynucleotides are released from the synthesis surface and stored in an alternative environment (e.g., storage container). Once released, the polynucleotides, in whole or in part, are sequenced 309, subject to decryption 310 to convert nucleic sequence back to digital sequence. The digital sequence is then assembled 311 to obtain an alignment encoding for the original item of information.

### Nucleic Acid Based Information Storage

Provided herein are devices, compositions, systems, and methods for nucleic acid-based information (data) storage. In some instances, biomolecules that have been synthesized and/or extracted from a substrate using the methods and compositions described herein may encode information for DNA data storage. A biomolecule such as a DNA molecule provides a suitable host for storage of information, such as digital information, in-part due to its stability over time and capacity for enhanced information coding, as opposed to traditional binary information coding. In addition, a biomolecule such as a DNA molecule can provide high volumetric storage density. In a first step, a digital sequence encoding an item of information (e.g., digital information in a binary code for processing by a computer) is received. The digital sequence can comprise a first plurality of symbols, such a binary, octal, decimal, or hexadecimal data. An encryption scheme is applied to convert the digital sequence from the first string of symbols to a second string of symbols. The second string of symbols can comprise an alternative representation to the first string of symbols. In some examples, the second string of symbols comprises a nucleic acid sequence.

Once an item of information is converted to a nucleic acid sequence, the nucleic acids can be synthesized. A surface material for nucleic acid extension, a design for loci for nucleic acid extension (aka, arrangement spots), and reagents for nucleic acid synthesis are selected. The surface of a structure is prepared for nucleic acid synthesis. De novo polynucleotide synthesis is then performed. The synthesized polynucleotides can be extracted, in whole or in part, using the systems, devices, methods, or platforms provided herein. The synthesized polynucleotides are stored in a structure and, in some cases, are available for subsequent release, in whole or in part. The synthesized polynucleotides may be stored in a structure suitable for long term storage (e.g., weeks, months, years, etc.). A structure suitable for long term storage may be identifiable and/or capable of being catalogues, such as, for example, using a tag (e.g., barcode or tag). Once released, the polynucleotides, in whole or in part, are sequenced, subject to decryption to convert nucleic sequence back to digital sequence. The digital sequence is then assembled to obtain an alignment encoding for the original item of information.

### Items of Information

Optionally, an early step of data storage process disclosed herein includes obtaining or receiving one or more items of information in the form of an initial code. In some instances, the items of information are encoded as a plurality of polynucleotides that have been extracted from a substrate, using systems, methods, platforms, or devices provided herein. Items of information (e.g., digital information) include, without limitation, text, audio, and visual information. Exemplary sources for items of information include, without limitation, books, periodicals, electronic databases, medical records, letters, forms, voice recordings, animal recordings, biological profiles, broadcasts, films, short videos, emails, bookkeeping phone logs, internet activity logs, drawings, paintings, prints, photographs, pixelated graphics, and software code. Exemplary biological profile sources for items of information include, without limitation, gene libraries, genomes, gene expression data, and protein activity data. Exemplary formats for items of information include, without limitation, .txt, .PDF, .doc, .docx, .ppt, .pptx, .xls, .xlsx, .rtf, .jpg, .gif, .psd, .bmp, tiff, .png, and .mpeg. The amount of individual file sizes encoding for an item of information, or a plurality of files encoding for items of information, in digital format include, without limitation, up to 1024 bytes (equal to 1 KB), 1024 KB (equal to 1 MB), 1024 MB (equal to 1 GB), 1024 GB (equal to 1 TB), 1024 TB (equal to 1PB), 1 exabyte, 1 zettabyte, 1 yottabyte, 1 xenottabyte or more. In some instances, an amount of digital information is at least 1 gigabyte (GB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 gigabytes. In some instances, the amount of digital information is at least 1 terabyte (TB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 terabytes. In some instances, the amount of digital information is at least 1 petabyte (PB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 petabytes. In some instances, the digital information does not contain genomic data acquired from an organism. Items of information in some instances are encoded. Non-limiting encoding method examples include 1 bit/base, 2 bit/base, 4 bit/base or other encoding method.

### Sequencing

Polynucleotides are extracted and/or amplified from surfaces where they are synthesized or stored. After extraction and/or amplification of polynucleotides from the surface of a structure, suitable sequencing technology may be employed to sequence the polynucleotides. In some cases, the DNA sequence is read on the substrate or within a feature of a structure. In some cases, the polynucleotides stored on the substrate are extracted, optionally assembled into longer polynucleotides and then sequenced. The polynucleotides may be extracted from the substrate using systems and methods described herein.

Polynucleotides synthesized and stored on the structures described herein encode data that can be interpreted by reading the sequence of the synthesized polynucleotides and converting the sequence into binary code readable by a computer. In some cases, the sequences require assembly, and the assembly step may need to be at the nucleic acid sequence stage or at the digital sequence stage.

Provided herein are detection systems comprising a device capable of sequencing stored polynucleotides, either directly on the synthesis structure and/or after removal from the main structure (e.g., synthesis structure, storage structure, etc.). In cases where the synthesis structure is a reel-to-reel tape of flexible material, the detection system comprises a device for holding and advancing the structure through a detection location and a detector disposed proximate the detection location for detecting a signal originated from a section of the tape when the section is at the detection location. In some instances, the signal is indicative of a presence of a polynucleotide. In some instances, the signal is indicative of a sequence of a polynucleotide (e.g., a fluorescent signal). In some instances, information encoded within polynucleotides on a continuous tape is read by a computer as the tape is conveyed continuously through a detector operably connected to the computer. In some instances, a detection system comprises a computer system comprising a polynucleotide sequencing device, a database for storage and retrieval of data relating to polynucleotide sequence, software for converting DNA code of a polynucleotide sequence to binary code, a computer for reading the binary code, or any combination thereof.

Provided herein are sequencing systems that can be integrated into the devices described herein. Various methods of sequencing are well known in the art and comprise “base calling” wherein the identity of a base in the target polynucleotide is identified. In some instances, polynucleotides synthesized using the methods, devices, compositions, and systems described herein are sequenced after cleavage from the synthesis surface. In some instances, sequencing occurs during or simultaneously with polynucleotide synthesis, wherein base calling occurs immediately after or before extension of a nucleoside monomer into the growing polynucleotide chain. Methods for base calling include measurement of electrical currents/voltages generated by polymerase-catalyzed addition of bases to a template strand. In some instances, synthesis surfaces comprise enzymes, such as polymerases. In some instances, such enzymes are tethered to electrodes or to the synthesis surface. In some instances, enzymes comprise terminal deoxynucleotidyl transferases, or variants thereof.

In some instances, the polynucleotides cleaved from a substrate surface or the amplified polynucleotides can be processed by techniques such as conventional or massively parallel sequencing. The sequencing can be done via various methods available in the field, e.g., methods involving incorporating one or more chain-terminating nucleotides, e.g., Sanger Sequencing method that can be performed by, e.g., SeqStudio® Genetic Analyzer from Applied Biosystems. In other embodiments, the sequencing can include performing a Next Generation Sequencing (NGS) method, e.g., primer extension followed by semiconductor-based detection (e.g., Ion Torrent™ systems from Thermo Fisher Scientific) or via fluorescent detection (e.g., Illumina systems).

### Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate. The computer systems may also be programmed to independently address one or more regions of a solid support, such as those provided herein.

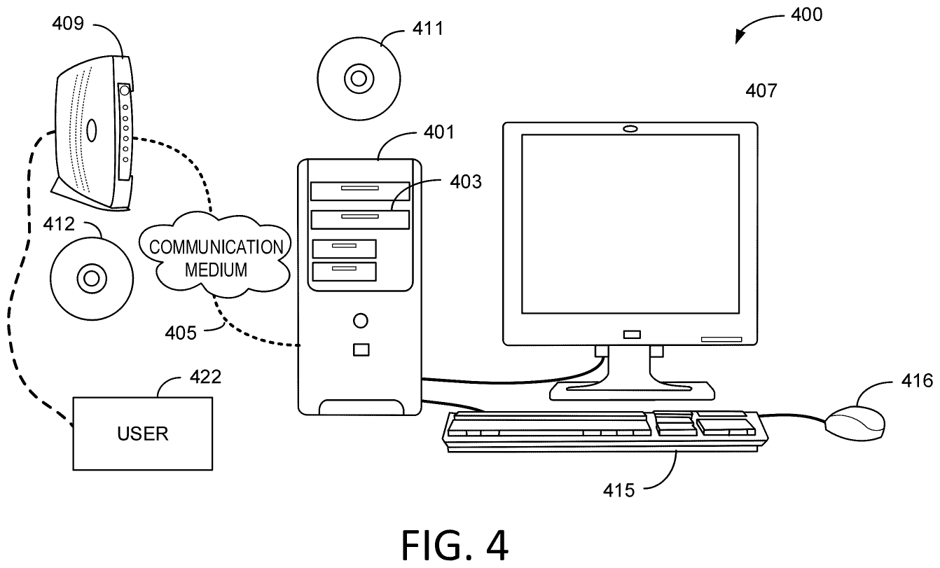

The computer system 400 illustrated in FIG. 4 may be understood as a logical apparatus that can read instructions from media 411 and/or a network port 405, which can optionally be connected to server 409 having fixed media 412. The system, such as shown in FIG. 4 can include a CPU 401, disk drives 403, optional input devices such as keyboard 415 and/or mouse 416 and optional monitor 407. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 422 as illustrated in FIG. 4.

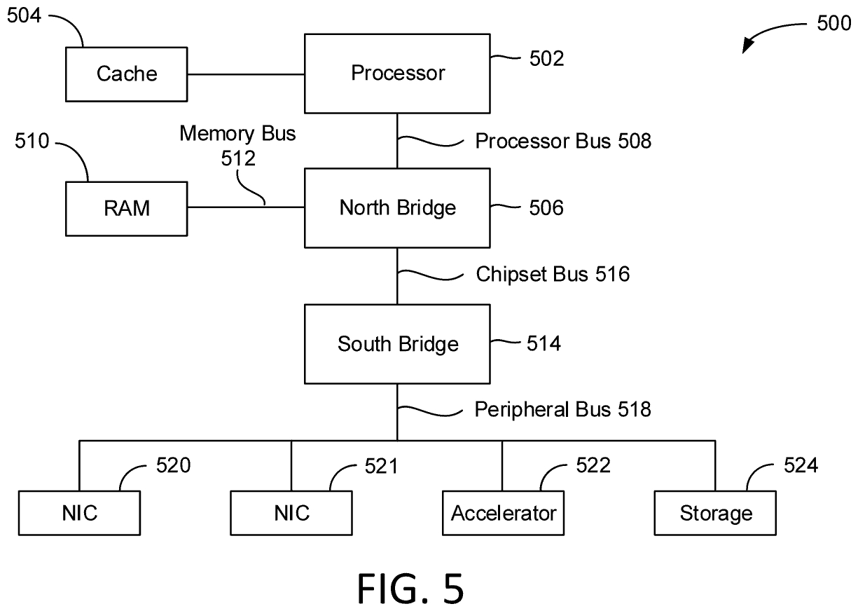

FIG. 5 is a block diagram illustrating a first example architecture of a computer system 500 that can be used in connection with example instances of the present disclosure. As depicted in FIG. 5, the example computer system can include a processor 502 for processing instructions. Non-limiting examples of processors include: Intel Xeon™ processor, AMD Opteron™ processor, Samsung 32-bit RISC ARM 1176JZ (F)-S v1.0TM processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 5, a high speed cache 504 can be connected to, or incorporated in, the processor 502 to provide a high speed memory for instructions or data that have been recently, or are frequently, used by processor 502. The processor 502 is connected to a north bridge 506 by a processor bus 508. The north bridge 506 is connected to random access memory (RAM) 510 by a memory bus 512 and manages access to the RAM 510 by the processor 502. The north bridge 506 is also connected to a south bridge 514 by a chipset bus 516. The south bridge 514 is, in turn, connected to a peripheral bus 518. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 518. In some alternative architectures, the functionality of the north bridge can be incorporated into the processor instead of using a separate north bridge chip. In some instances, system 500 can include an accelerator card 522 attached to the peripheral bus 518. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 524 and can be loaded into RAM 510 and/or cache 504 for use by the processor. The system 500 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present disclosure. In this example, system 500 also includes network interface cards (NICs) 520 and 521 connected to the peripheral bus for providing network interfaces to external storage, such as Network Attached Storage (NAS) and other computer systems that can be used for distributed parallel processing.

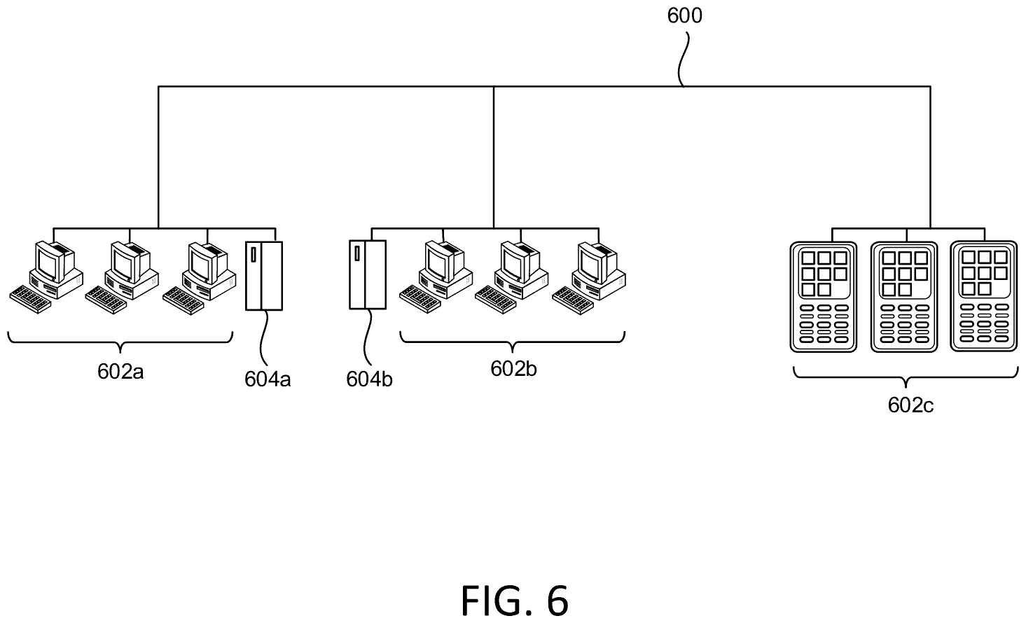

FIG. 6 is a diagram showing a network 600 with a plurality of computer systems 602a, and 602b, a plurality of cell phones and personal data assistants 602c, and Network Attached Storage (NAS) 604a, and 604b. In example instances, systems 602a, 602b, and 602c can manage data storage and optimize data access for data stored in Network Attached Storage (NAS) 604a and 604b. A mathematical model can be used for the data and be evaluated using distributed parallel processing across computer systems 602a, and 602b, and cell phone and personal data assistant systems 602c. Computer systems 602a, and 602b, and cell phone and personal data assistant systems 602c can also provide parallel processing for adaptive data restructuring of the data stored in Network Attached Storage (NAS) 604a and 604b. FIG. 6 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

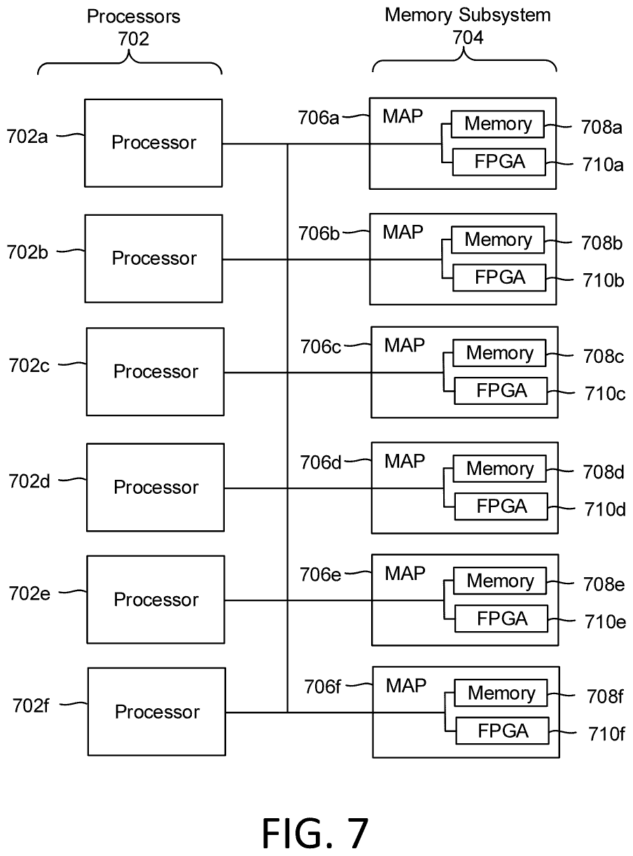

FIG. 7 is a block diagram of a multiprocessor computer system using a shared virtual address memory space in accordance with an example instance. The system includes a plurality of processors 702a-f that can access a shared memory subsystem 704. The system incorporates a plurality of programmable hardware memory algorithm processors (MAPs) 706a-f in the memory subsystem 704. Each MAP 706a-f can comprise a memory 708a-f and one or more field programmable gate arrays (FPGAs) 710a-f. The MAP provides a configurable functional unit and particular algorithms or portions of algorithms can be provided to the FPGAs 710a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 708a-f, allowing it to execute tasks independently of, and asynchronously from the respective microprocessor 702a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 5, system on chips (SOCs), application specific integrated circuits (ASICs), or other processing and logic elements. For example, the Set Processor and Optimizer can be implemented with hardware acceleration through the use of a hardware accelerator card, such as accelerator card 522 illustrated in FIG. 5.

### EXAMPLES

The following examples are set forth to illustrate more clearly the principles and practice of embodiments disclosed herein to those skilled in the art and are not to be construed as limiting the scope of any claimed embodiments. Unless otherwise stated, all parts and percentages are on a weight basis.

### Example 1: Single Strand Chain Extension Using dN6P Substrates and TdT Enzyme

TdT was used for single strand extension. dNTP-TdT conjugates were constructed with modification following the general methods of Palluk, et al., 2018, “De novo DNA synthesis using polymerase-nucleotide conjugates,” Nat. Biotechnol. 36, 645-650. Linkers that do not leave a scar were incorporated.

Briefly, TdT was incubated with a single stranded DNA, manganese, and dA6P (deoxyadenosine hexaphosphate) substrate. No protecting group was used on 3′ end, resulting in multiple additions of dA.

TdT cysteine variant NTT-1 was also used for single strand extension. Using such NTT-TIDES conjugates, NTT-1 was found to exhibit extension activity. Enzymatic synthesis was then performed on a surface. Briefly, reverse phosphoramidites (phosphoramidite on the 5′ hydroxyl monomer) were used as was diethylamine to gently remove cyanoethyl group, leaving linker attachment in place. dT was also used, resulting in successful extension. Single strand chain extension was also performed using dATPs and dA6Ps.

### Example 2: Substrate Cleavage Via Uracil

Polynucleotide synthesis is performed on a surface. In enzymatic DNA synthesis, the extension generally occurs 5′ to 3′ and the synthesis starts with a native or native-like nucleic acid strand as a substrate for the terminal transferase (e.g., TdT). Generating this strand on surfaces in some instances occurs through chemical synthesis using reverse thymidine phosphoramidites in 5′ to 3′ direction. This strand in some instances is treated with base such as diethylamine or other substituted amine to remove cyanoethyl protecting groups leaving a tethered native DNA strand. This strand may also be prepared with a 5′-modification that can then be reacted with the surface. This conjugation could be thiol/maleimide, NHS ester/amine, copper assisted or copper-free Huisgen cycloaddition, TCO/tetrazine. This strand can then be acted on by the terminal transferase, however, resulting cleavage of the entire strand in some instances leaves an oligothymidine “stilt”. Described in this example are methods of cleavage of the enzymatic-derived oligonucleotide from the chemically-synthesized “stilt”.

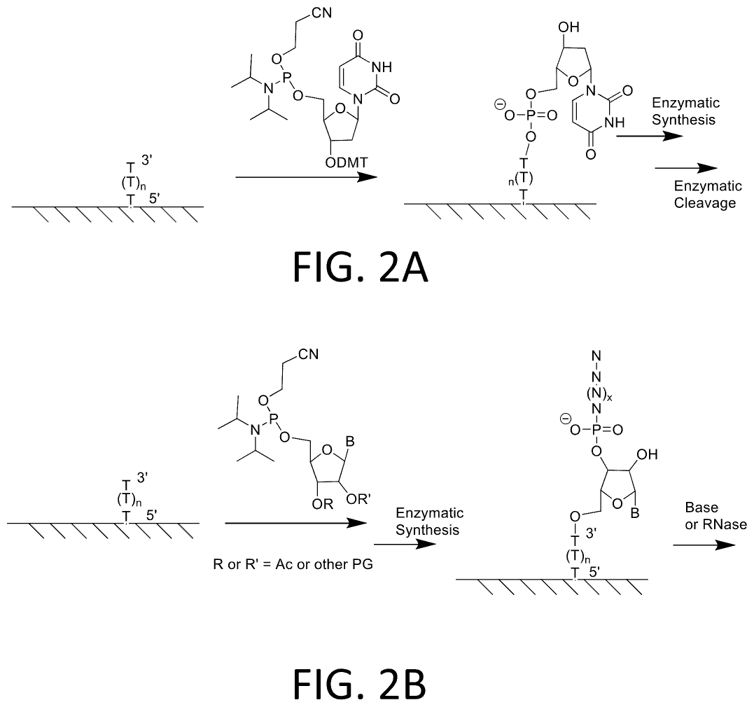

If deoxy uracil is chemically synthesized as the last nucleotide at 3′-end of the stilt enzymatic synthesis may begin as TdT will recognize this nucleotide and extend the chain (FIG. 2A). After the desired sequence is synthesized enzymatically, treatment with uracil DNA glycosylase excises the base leaving an aldehydic anomeric carbon. This sugar can then be treated with mild base to break the strand leaving 5′ and 3′ phosphate strands. Alternatively, after base excision, treatment with an apurinic/apyrimidinic (AP) endonuclease cleaves the strand. AP classes I-IV may be used to generate alternately phosphorylated or unphosphorylated 3′- and 5′-ends of the cleaved strands.

Base excision repair (BER) enzymes may be used for different endogenous targets. These targets are “damaged” bases such as 3-methyladenine, 8-oxo-guanine, 2,6-diamino-4-hydroxy-5-formamidopyrimidine (FapyG), 4,6-diamino-5-formamidopyrimidine (FapyA), 5-hydroxyuracil, 5-hydroxymethyluracil, and 5-formyluracil. These bases may be incorporated using phosphoramidite chemistry with phosphoramidites that contain labile base-protecting groups that may be cleaved before enzymatic synthesis begins. Alkylpurines may additionally be excised by alkylpurine glycosylases C and D (AlkC, AlkD). Bi-functional DNA glycosylases may also be used such as OGG1, NTH1, NEIL1-3, and their homologues so there is no need for a secondary enzymatic treatment. Endonuclease V may be used to cleave at an inserted inosine. In some instances, the site where cleavage occurs is further from the start of the enzymatic synthesis.

### Example 3: Enzymatic Substrate Cleavage Via Uracil

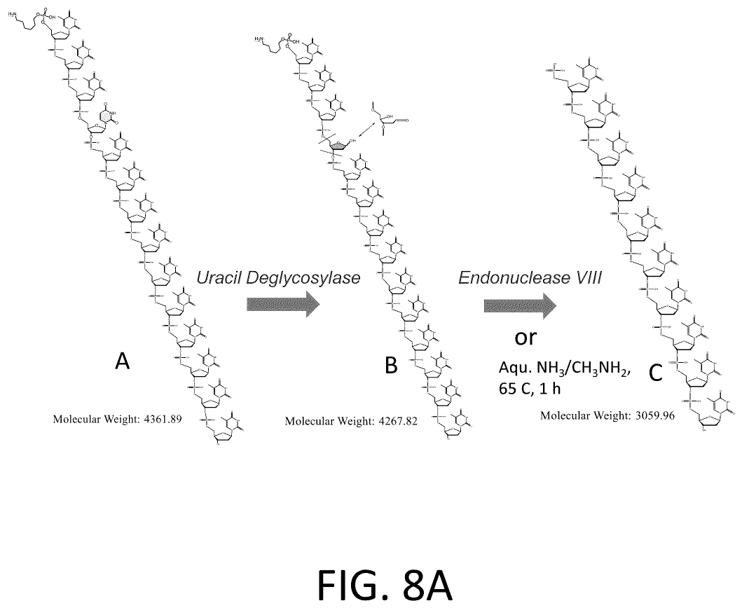

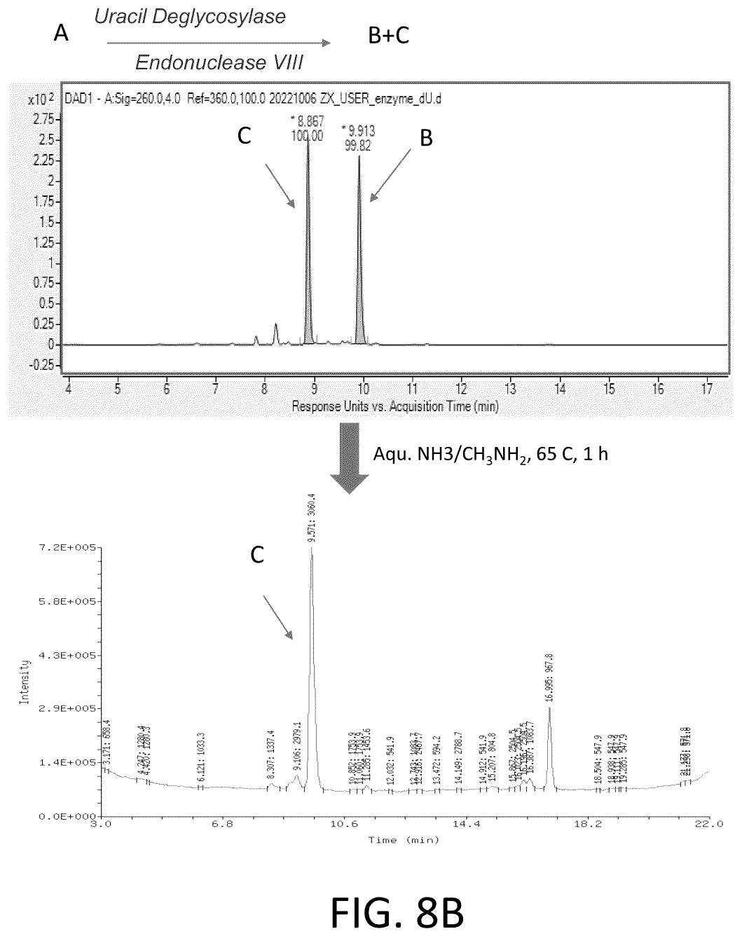

The general procedures of Example 2 were followed to synthesize a polynucleotide with deoxy uracil (A in FIG. 8A). After the desired sequence was synthesized, treatment with uracil deglycosylase excised the base leaving an aldehydic anomeric carbon (B in FIG. 8A). After base excision, treatment with endonuclease VIII was used to cleave the strand (C in FIG. 8A). The results were analyzed via LCMS, which showed both intermediate product B and the cleaved product C (FIG. 8B, top).

Subsequent treatment was performed using an aqueous base (NH₃/CH₃NH₂) and heat (at 65 degrees Celsius) for one hour. The results were analyzed, which showed an increase in yield of the cleaved product (FIG. 8B, bottom).

### Example 4: Substrate Cleavage Via Ribonucleotide

The general procedures of Example 2 are followed with modification: an RNA nucleotide may also be incorporated at 3′-end of the stilt (FIG. 2B). Treatment of this DNA/RNA hybrid with basic conditions results in a 3′-cyclic phosphate at the stilt and a 5′-OH on the enzymatically synthesized strand. In many of these embodiments a complementary strand to the region surrounding the excision site is required for many of these enzymatic cleavage routes. Mismatches can also be introduced in this way providing T:G mismatches that are excised by thymidine DNA glycosylase (TDG) and/or methyl-CpG-binding domain protein 4 (MBD4).

In some embodiments several RNA bases may be added to the end of the stilt. Addition of a DNA complement to the RNA region in the presence of RNase H results in cleavage of the synthesized nucleic acid from the surface. Un-cleaved RNA still present can later be removed enzymatically or through incubation under basic conditions. By hybridization of a DNA complement to the stilt region restriction endonucleases such as BamHI, EcoRI, EcoRV, HindIII, and HaeIII amongst others may be used to cleave specific enzymatically synthesized sequence selectively.

### Example 5: Substrate Cleavage Via Electrochemically Generated Acid or Base

The general procedures of Example 2 are followed with modification: cleavage of the polynucleotide from the surface is effected by use of an acid or base sensitive linker which connects the polynucleotide to the surface.

In one embodiment, acid is generated my applying a potential to a solution containing a mixture of benzoquinone and hydroquinone. An acid labile linker may compose an aldol or tetrahydrofuran based-linker, trityl or variously substituted trityl-based linker.

In another embodiment, base is generated with a solution of unsubstituted or 1,6 or 2,7 disubstituted phenazine or tetrasubstituted phenazine with their respective corresponding hydrophenazine compounds. Protic solvent in solution can be primary, secondary or tertiary alcohols. Deprotonation of these compounds results in a species that can initiate cleavage from the surface. These molecules could also be phenolic, cresolic or catecholic in nature. The molecules could also be amine based whereby the pKa of the amino proton can be manipulated by various substitutions which include but are not limited to trifluoromethylsulfonyl, hexafluoropropyl, trifluoromethyl, pentafluorophenyl or nitrophenyl, optionally containing halogens varying in number to manipulate the pKa of the respective compounds.

### Example 6: Substrate Cleavage Using Redox-Active Linker

The general procedures of Example 5 are followed with modification: the linker comprises a redox-active chemical group. The linker could be cleaved by a (3-elimination reaction in a similar way to decyanoethylation of the phosphate backbone in standard phosphonamidite chemistry. This linker could contain an electron withdrawing functional group such as but not limited to sulfone, fluorine(s), nitro group, sulfonyl or cyano. The linker could be cleaved by unmasking an internal nucleophile that ‘bites back’ on itself to result in dissociation of the biological on non-biological molecule of interest. The linker could have a levulinyl fragment or component. The linker could be an ester derivative of hydroquinone-O,O-diacetic acid (Q-linker). The linker could be a variously alkyl-substituted silane which could be cleaved by electrochemical production of an alkoxide. The linker could be subject to cleavage by an active metal center that may be generated by oxidation or reduction of a metal center. This metal could be but in but not limited to groups 8-10 of the periodic table. The linker could contain an organoborane that could be cleaved through the mechanism of oxidative elimination followed by reductive elimination (think Suzuki coupling and related). The linker could be composed of an aryl or alkyl sulfonate that could oxidatively add to an electrochemically-generated metal center. The linker could itself contain a transition metal complex that under oxidation or reduction a structural change results in release of the ligand-modified biomolecule. The linker could comprise one or more embedded or pendant redox-active molecules such as quinone, imide, carbazole viologen, organosulfur compounds, triphenylamine, ferrocene, or radical compounds such as nitroxyl, phenoxyl, and verdazyl groups, with stable charge/discharge voltage and high reactivity. The biomolecule may be tethered to the surface by a ligation that can be competed off by deprotonation or otherwise demasking a ligand with a lower kD in respect to the metal center. These metal complexes can be anchored to the surface of the device or can be floating free in solution.

### Example 7: Substrate Cleavage Using Photolabile Linker

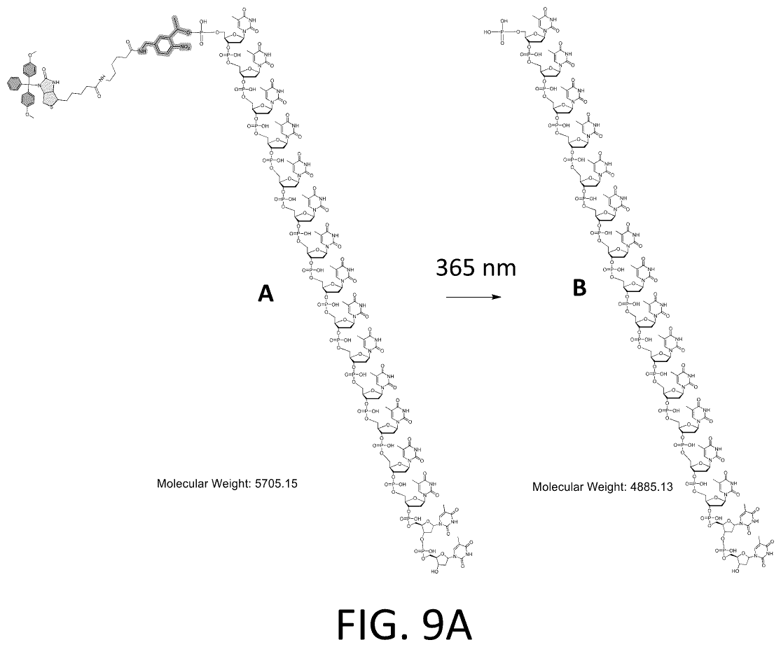

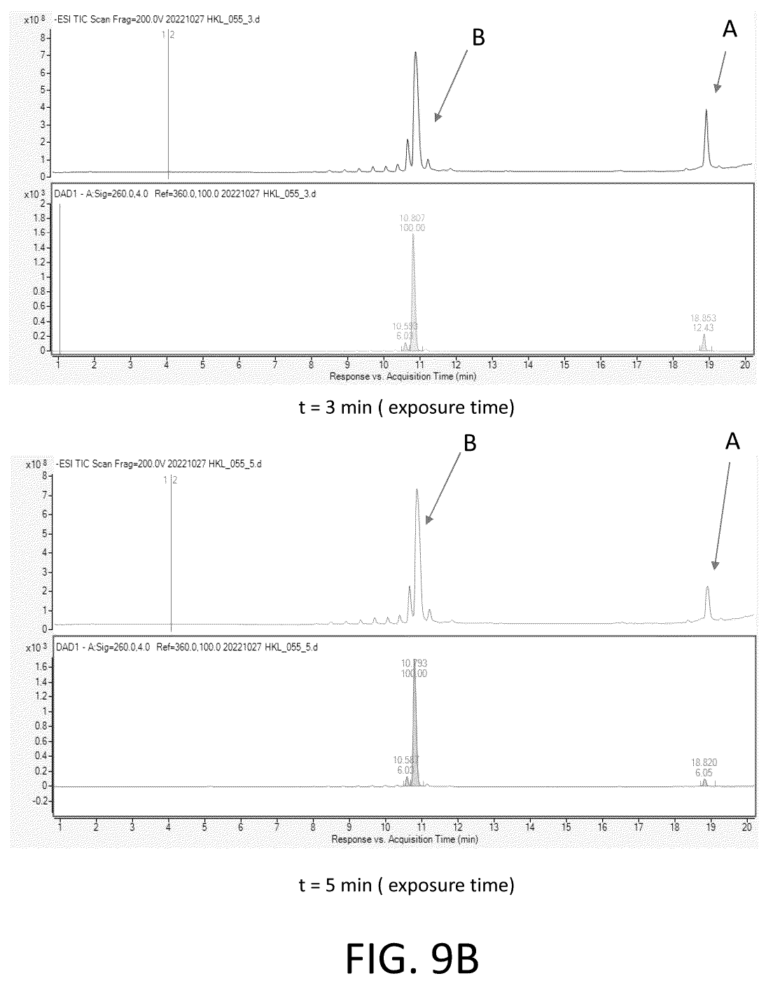

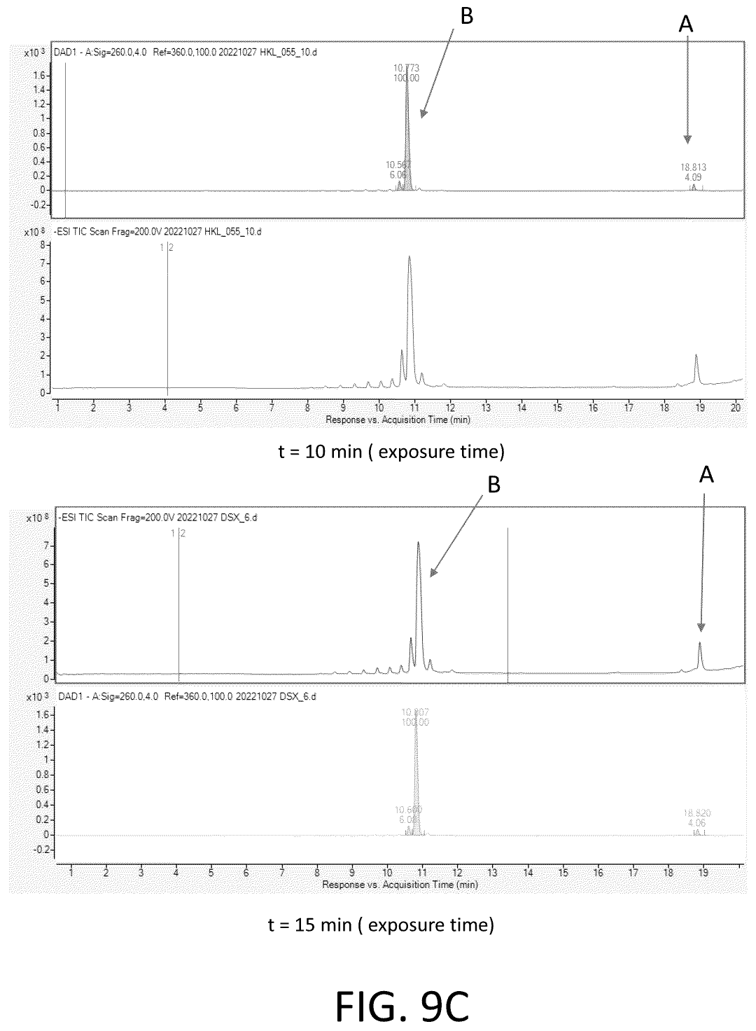

The general procedures of Example 2 are followed with modification: Polynucleotide synthesis is performed on a surface. This strand may also be prepared with a 5′-modification that can then reacted with a suitably modified surface. This conjugation could be thiol/maleimide, NHS ester/amine, copper assisted or copper-free Huisgen cycloaddition, TCO/tetrazine. The support linker contains one or more photo-cleavable units. In some embodiments, the photo-cleavable linker is an orthonitrobenzyl-based linker, phenacyl linker, alkoxybenzoin linker, chromium arene complex linker, NpSSMpact linker, or pivaloylglycol linker. In some embodiments, the photo-cleavable linker can be cleaved by irradiating the linker at about 312 nm, 365 nm or at about 405 nm (e.g., FIG. 9A). In enzymatic DNA synthesis, the extension generally occurs 5′ to 3′ and the synthesis starts with a native or native-like nucleic acid strand as a substrate for the terminal transferase (e.g., TdT). Generating this strand on surfaces in some instances occurs through chemical synthesis using reverse thymidine phosphoramidites in 5′ to 3′ direction. This strand in some instances is treated with base such as diethylamine or other substituted amine to remove cyanoethyl protecting groups leaving a tethered native DNA strand. This strand can then be acted on by the terminal transferase, however, resulting cleavage of the entire strand in some instances leaves an oligothymidine “stilt”. Further described herein are methods of cleavage of the enzymatic-derived oligonucleotide from the chemically-synthesized “stilt” at the photolabile site which was introduced into the support linker.

### Example 8: Substrate Cleavage Using an Orthonitrobenzyl-Based Photolabile Linker

The general procedures of Example 7 were followed with an orthonitrobenzyl-based linker in the support linker (FIG. 9A). The sample contained 1 uM of the polynucleotide (A) with the photolabile linker in 100 uL pH 7.0 buffer. The sample was exposed to 365 nm wavelength to cleave the linker (B) and analyzed via LCMS.

The sample was irradiated for 3 minutes (FIG. 9B, top), 5 minutes (FIG. 9B, bottom), 10 minutes (FIG. 9C, top), and 15 minutes (FIG. 9C, bottom). As shown in the LCMS chromatograms, as the exposure time increased, the peak corresponding to the cleaved product (B) increased and the peak corresponding to the uncleaved polynucleotide (A) decreased. About 95% cleavage of the orthonitrobenzyl-based linker was achieved after exposure time of about 10 minutes (FIG. 9C, top).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method for cleaving a polynucleotide, comprising:
(a) synthesizing a plurality of polynucleotides each comprising one or more bases susceptible to enzymatic cleavage;
(b) exposing the plurality of polynucleotides to one or more enzymes; and
(c) treating the plurality of polynucleotides in an aqueous base at a temperature of about 55 degrees Celsius to 75 degrees Celsius.

2. The method of claim 1, wherein exposing the plurality of polynucleotides to the one or more enzymes comprises exposing the plurality of polynucleotides to a first enzyme of the one or more enzymes.

3. The method of claim 2, wherein exposing the plurality of polynucleotides to the one or more enzymes further comprises exposing the plurality of polynucleotides to a second enzyme of the one or more enzymes.

4. The method of claim 3, wherein the first enzyme and the second enzyme are different enzymes.

5. The method of claim 1, wherein synthesizing comprises enzymatic synthesis or chemical synthesis.

6. The method of claim 1, wherein synthesizing comprises synthesizing the plurality of polynucleotides on a solid support.

7. The method of claim 6, wherein the plurality of polynucleotides are attached to a surface of the solid support via a support linker.

8. The method of claim 7, wherein the support linker comprises a stilt.

9. The method of claim 8, wherein the stilt comprises thymidine.

10. The method of claim 1, wherein the one or more bases comprises deoxy uracil.

11. The method of claim 1, wherein the one or more enzymes comprises one or more of uracil DNA glycosylase, apurinic/apyrimidinic (AP) endonuclease, alkylpurine glycosylases C and D, OGG1, NTH1, NEIL1-3, Endonuclease V, or endonuclease VII.

12. The method of claim 1, wherein the plurality of polynucleotides are treated in the aqueous base for about one hour.

13. The method of claim 1, wherein the temperature is about 65 degrees Celsius.

14. A method for cleaving a polynucleotide, comprising:
(a) synthesizing a plurality of polynucleotides on a surface of a solid support, wherein the plurality of polynucleotides are attached to the surface via a support linker; and
(b) irradiating the plurality of polynucleotides.

15. The method of claim 14, wherein synthesizing comprises enzymatic synthesis or chemical synthesis.

16. The method of claim 14, wherein the support linker comprises a stilt.

17. The method of claim 16, wherein the stilt comprises thymidine.

18. The method of claim 14, wherein the support linker comprises photo-cleavable linker.

19. The method of claim 18, wherein the photo-cleavable linker comprises an orthonitrobenzyl-based linker, phenacyl linker, alkoxybenzoin linker, chromium arene complex linker, NpSSMpact linker, or pivaloylglycol linker.

20. The method of claim 18, wherein the photo-cleavable linker is cleaved by irradiating the support linker at about 312 nm, 365 nm or 405 nm.

21. The method of claim 18, wherein the photo-cleavable linker is irradiated for about 1 minutes to about 15 minutes.

22. A method of synthesizing a polynucleotide, comprising:
(a) contacting a polynucleotide with a complex according to the following formula: 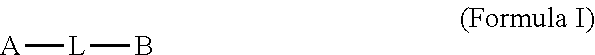
wherein:
A comprises a polymerase;
B comprises a nucleotide; and
L comprises a chemical linker that covalently links the polymerase to a terminal phosphate group of the nucleotide, wherein the polymerase is configured to catalyze covalent addition of the nucleotide onto a 3′ hydroxyl of a polynucleotide, and subsequent extension of the polynucleotide from a surface of a solid support, wherein the polynucleotide is attached to the surface via a support linker; and
(b) cleaving the polymerase from the polynucleotide, wherein the cleaving does not leave a part of the linker on the polynucleotide.

23. The method of claim 22, wherein the method further comprises cleaving the polynucleotide from the solid support.

24. The method of claim 23, wherein the method further comprises cleaving the polynucleotide from the solid support with an enzyme.

25. The method of claim 22, wherein the support linker comprises a stilt.

26. The method of claim 25, wherein the stilt comprises thymidine.

27. The method of claim 22, wherein the support linker comprises uracil.

28. The method of claim 22, wherein the support linker comprises one or more of 3-methyladenine, 8-oxo-guanine, oxo-inosine, 2,6-diamino-4-hydroxy-5-formamidopyrimidine (FapyG), 4,6-diamino-5-formamidopyrimidine (FapyA), 5-hydroxyuracil, 5-hydroxymethyluracil, or 5-formyluracil.

29. The method of claim 24, wherein the enzyme comprises one or more of uracil DNA glycosylase, apurinic/apyrimidinic (AP) endonuclease, alkylpurine glycosylases C and D, OGG1, NTH1, NEIL1-3, or Endonuclease V.

30. The method of claim 22, wherein the support linker comprises one or more ribonucleosides.

31. The method of claim 30, wherein the one or more ribonucleosides comprise protecting groups at one or both of the 2′ and 3′ OH positions.

32. The method of claim 31, wherein the protecting groups comprise acetyl, benzoyl, trimethylsilyl, TBDMS, TOM, or levulinyl.

33. The method of claim 24, wherein the enzyme comprises RNase H.

34. The method of claim 22, wherein the method further comprises hybridizing a complementary or partially complementary polynucleotide to the support linker.

35. The method of claim 24, wherein the enzyme comprises one or more of thymidine DNA glycosylase (TDG) and methyl-CpG-binding domain protein 4 (MBD4).

36. The method of claim 24, wherein the enzyme comprises one or more of BamHI, EcoRI, EcoRV, HindIII, and HaeIII.

37. The method of claim 22, wherein steps a)-b) are repeated to produce an extended polynucleotide.

38. The method of claim 22, wherein the extended polynucleotide comprises at least about 50 nucleotides.

39. The method of claim 22, wherein the polymerase is a template-independent polymerase.

40. The method of claim 39, wherein the polymerase is terminal deoxynucleotidyl transferase (TdT) or polymerase theta.

41. The method of claim 22, wherein the chemical linker is an acid-labile linker, a base-labile linker, a pH-sensitive linker, an amine-to-thiol crosslinker, thiomaleamic acid linker, or a photo-cleavable linker.

42. The method of claim 41, wherein the photo-cleavable linker is selected from the group consisting of orthonitrobenzyl-based linker, phenacyl linker, alkoxybenzoin linker, chromium arene complex linker, NpSSMpact linker, pivaloylglycol linker, and any combination thereof.

43. The method of claim 22, wherein the chemical linker is selected from the group consisting of a silyl linker, an alkyl linker, a polyether linker, a polysulfonyl linker, a polysulfoxide linker, and any combination thereof.

44. The method of claim 22, wherein the nucleotide comprises at least 3 phosphate groups.

45. The method of claim 22, wherein the nucleotide is selected from the group consisting of nucleoside triphosphate, nucleoside tetraphosphate, nucleoside pentaphosphate, nucleoside hexaphosphate, nucleoside heptaphosphate, nucleoside octaphosphate, nucleoside nonaphosphate, and any combination thereof.

46. The method of claim 45, wherein the nucleotide is selected from the group consisting of deoxyadenosine triphosphate (dATP), deoxyguanosine triphosphate (dGTP), deoxycytidine triphosphate (dCTP), deoxythymidine triphosphate (dTTP), deoxyadenosine tetraphosphate, deoxyguanosine tetraphosphate, deoxycytidine tetraphosphate, deoxythymidine tetraphosphate, deoxyadenosine pentaphosphate, deoxyguanosine pentaphosphate, deoxycytidine pentaphosphate, deoxythymidine pentaphosphate, deoxyadenosine hexaphosphate, deoxyguanosine hexaphosphate, deoxycytidine hexaphosphate, deoxythymidine hexaphosphate, and any combination thereof.

47. A method of synthesizing a polynucleotide, comprising:
(a) contacting a polynucleotide with a complex according to the following formula: 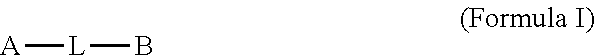
wherein:
A comprises a polymerase;
B comprises a nucleotide; and
L comprises a chemical linker that covalently links the polymerase to a terminal phosphate group of the nucleotide, wherein the polymerase is configured to catalyze covalent addition of the nucleotide onto a 3′ hydroxyl of a polynucleotide, and subsequent extension of the polynucleotide from a surface of a solid support, wherein the polynucleotide is attached to the surface via a support linker; and
(b) extending the polynucleotide by addition of the nucleotide, wherein the addition of the nucleotide results in cleavage between the chemical linker and the nucleotide; and
(c) cleaving the polymerase from the polynucleotide, wherein the cleaving does not leave a part of the linker on the polynucleotide.

48. The method of claim 47, wherein the method further comprises cleaving the polynucleotide from the solid support.

49. The method of claim 48, wherein the method further comprises cleaving the polynucleotide from the solid support using a chemical reaction.

50. The method of claim 48, wherein cleavage of the polynucleotide is independently addressable.

51. The method of claim 49, wherein the chemical reaction comprises acid, base, or electrochemistry.

52. The method of claim 48, wherein the method further comprises generation of acid at a region of the surface.

53. The method of claim 52, wherein the acid is generated by applying a potential to a solution containing a mixture of benzoquinone and hydroquinone, or derivatives thereof.

54. The method of claim 48, wherein the support linker comprises an aldol, tetrahydrofuran, or trityl group.

55. The method of claim 48, wherein the method further comprises generation of base at a region of the surface.

56. The method of claim 55, wherein the base is generated by applying a potential to a solution containing (1) an arene or a heteroarene; and (2) a protic solvent.

57. The method of claim 56, wherein the arene or the heteroarene comprises one or more of substituted or unsubstituted azobenzene, hydrabenzene, azophenanthrene, azonapthalene, or azopyridine.

58. The method of claim 56, wherein the protic solvent comprises an alcohol.

59. The method of claim 55, wherein the base is generated by applying a potential to a solution containing unsubstituted, 1,6 or 2,7 disubstituted phenazine, or tetrasubstituted phenazine with their respective corresponding hydrophenazine compounds.

60. The method of claim 56, wherein the arene or the heteroarene comprises a phenolic, cresolic or catecholic group.

61. The method of claim 56, wherein the arene or the heteroarene comprises an amine.

62. The method of claim 56, wherein the arene or the heteroarene is substituted with one or more of trifluoromethylsulfonyl, hexafluoropropyl, trifluoromethyl, pentafluorophenyl, or nitrophenyl.

63. The method of claim 56, wherein the arene or the heteroarene is substituted with one or more halogens.

64. The method of claim 47, wherein the support linker comprises an ester.

65. The method of claim 48, wherein the support linker is cleaved by beta elimination.

66. The method of claim 65, wherein the support linker comprises an electron withdrawing group.

67. The method of claim 66, wherein the electron withdrawing group comprises sulfone, fluorine(s), nitro group, sulfonyl or cyano.

68. The method of claim 48, wherein the support linker comprises a latent nucleophile.

69. The method of claim 48, wherein the support linker comprises a levulinyl group.

70. The method of claim 48, wherein the support linker comprises hydroquinone-O,O-diacetic acid (Q-linker).

71. The method of claim 48, wherein the support linker comprises an alkyl-substituted silane.

72. The method of claim 48, wherein the method further comprises an electrochemical reaction.

73. The method of claim 72, wherein the support linker comprise a redox-active group.

74. The method of claim 72, wherein the support linker comprises a metal center.

75. The method of claim 74, wherein the metal center comprises a metal of any one of groups 8-10 of the periodic table.

76. The method of claim 72, wherein the support linker comprises an organoborane.

77. The method of claim 72, wherein the support linker comprises an aryl or an alkyl sulfonate.

78. The method of claim 72, wherein the support linker comprises a ligand.

79. The method of claim 78, wherein the support comprises a ligand binder.

80. The method of claim 47, wherein the method comprises cleaving the polynucleotide from the solid support with an enzyme.

81. The method of claim 47, wherein the support linker comprises a stilt.

82. The method of claim 81, wherein the stilt comprises thymidine.

83. The method of claim 47, wherein the support linker comprises uracil.

84. The method of claim 47, wherein the support linker comprises one or more of 3-methyladenine, 8-oxo-guanine, oxo-inosine, 2,6-diamino-4-hydroxy-5-formamidopyrimidine (FapyG), 4,6-diamino-5-formamidopyrimidine (FapyA), 5-hydroxyuracil, 5-hydroxymethyluracil, or 5-formyluracil.

85. The method of claim 80, wherein the enzyme comprises one or more of uracil DNA glycosylase, apurinic/apyrimidinic (AP) endonuclease, alkylpurine glycosylases C and D, OGG1, NTH1, NEIL1-3, Endonuclease V, or endonuclease VII.

86. The method of claim 80, further comprising treating the polynucleotide with an aqueous base, heating the polynucleotides, or a combination thereof.

87. The method of claim 86, wherein heating the polynucleotides comprises heating at a temperature of about 55 to 75 degrees Celsius.

88. The method of claim 47, wherein the support linker comprises one or more ribonucleosides.

89. The method of claim 88, wherein the one or more ribonucleosides comprise protecting groups at one or both of the 2′ and 3′ OH positions.

90. The method of claim 89, wherein the protecting groups comprise acetyl, benzoyl, trimethylsilyl, TBDMS, TOM, or levulinyl.

91. The method of claim 80, wherein the enzyme comprises RNase H.

92. The method of claim 47, wherein the method further comprises hybridizing a complementary or partially complementary polynucleotide to the support linker.

93. The method of claim 92, wherein the enzyme comprises one or more of thymidine DNA glycosylase (TDG) and methyl-CpG-binding domain protein 4 (MBD4).

94. The method of claim 92, wherein the enzyme comprises one or more of BamHI, EcoRI, EcoRV, HindIII, and HaeIII.

95. The method of claim 47, wherein steps a)-c) are repeated to produce an extended polynucleotide.

96. The method of claim 47, wherein the extended polynucleotide comprises at least about 10 nucleotides.

97. The method of claim 47, wherein the polymerase is a template-independent polymerase.

98. The method of claim 97, wherein the polymerase is terminal deoxynucleotidyl transferase (TdT) or polymerase theta.

99. The method of claim 47, wherein the chemical linker is an acid-labile linker, a base-labile linker, a pH-sensitive linker, an amine-to-thiol crosslinker, thiomaleamic acid linker, or a photo-cleavable linker.

100. The method of claim 99, wherein the photo-cleavable linker is selected from the group consisting of orthonitrobenzyl-based linker, phenacyl linker, alkoxybenzoin linker, chromium arene complex linker, NpSSMpact linker, pivaloylglycol linker, and any combination thereof.

101. The method of claim 47, wherein the chemical linker is selected from the group consisting of a silyl linker, an alkyl linker, a polyether linker, a polysulfonyl linker, a polysulfoxide linker, and any combination thereof.

102. The method of claim 47, wherein the nucleotide comprises at least 3 phosphate groups.

103. The method of claim 47, wherein the nucleotide is selected from the group consisting of nucleoside triphosphate, nucleoside tetraphosphate, nucleoside pentaphosphate, nucleoside hexaphosphate, nucleoside heptaphosphate, nucleoside octaphosphate, nucleoside nonaphosphate and any combination thereof.

104. The method of claim 103, wherein the nucleotide is selected from the group consisting of deoxyadenosine triphosphate (dATP), deoxyguanosine triphosphate (dGTP), deoxycytidine triphosphate (dCTP), deoxythymidine triphosphate (dTTP), deoxyadenosine tetraphosphate, deoxyguanosine tetraphosphate, deoxycytidine tetraphosphate, deoxythymidine tetraphosphate, deoxyadenosine pentaphosphate, deoxyguanosine pentaphosphate, deoxycytidine pentaphosphate, deoxythymidine pentaphosphate, deoxyadenosine hexaphosphate, deoxyguanosine hexaphosphate, deoxycytidine hexaphosphate, deoxythymidine hexaphosphate, and any combination thereof.

1. A method for cleaving a polynucleotide, comprising:
(a) synthesizing a plurality of polynucleotides each comprising one or more bases susceptible to enzymatic cleavage;
(b) exposing the plurality of polynucleotides to one or more enzymes; and
(c) treating the plurality of polynucleotides in an aqueous base at a temperature of about 55 degrees Celsius to 75 degrees Celsius.

2. The method of claim 1, wherein exposing the plurality of polynucleotides to the one or more enzymes comprises exposing the plurality of polynucleotides to a first enzyme of the one or more enzymes.

3. The method of claim 2, wherein exposing the plurality of polynucleotides to the one or more enzymes further comprises exposing the plurality of polynucleotides to a second enzyme of the one or more enzymes.

4. The method of claim 3, wherein the first enzyme and the second enzyme are different enzymes.

5. The method of claim 1, wherein synthesizing comprises enzymatic synthesis or chemical synthesis.

6. The method of claim 1, wherein synthesizing comprises synthesizing the plurality of polynucleotides on a solid support.

7. The method of claim 6, wherein the plurality of polynucleotides are attached to a surface of the solid support via a support linker.

8. The method of claim 7, wherein the support linker comprises a stilt.

9. The method of claim 8, wherein the stilt comprises thymidine.

10. The method of claim 1, wherein the one or more bases comprises deoxy uracil.

11. The method of claim 12, wherein the one or more enzymes comprises one or more of uracil DNA glycosylase, apurinic/apyrimidinic (AP) endonuclease, alkylpurine glycosylases C and D, OGG1, NTH1, NEIL1-3, Endonuclease V, or endonuclease VII.

12. (canceled)

13. The method of claim 1, wherein the temperature is about 65 degrees Celsius.

14. A method for cleaving a polynucleotide, comprising:
(a) synthesizing a plurality of polynucleotides on a surface of a solid support, wherein the plurality of polynucleotides are attached to the surface via a support linker; and
(b) irradiating the plurality of polynucleotides.

15. The method of claim 14, wherein synthesizing comprises enzymatic synthesis or chemical synthesis.

16. The method of claim 14, wherein the support linker comprises a stilt.

17. The method of claim 16, wherein the stilt comprises thymidine.

18. The method of claim 14, wherein the support linker comprises photo-cleavable linker.

19. The method of claim 18, wherein the photo-cleavable linker comprises an orthonitrobenzyl-based linker, phenacyl linker, alkoxybenzoin linker, chromium arene complex linker, NpSSMpact linker, or pivaloylglycol linker.

20. The method of claim 18, wherein the photo-cleavable linker is cleaved by irradiating the support linker at about 312 nm, 365 nm or 405 nm.

21. The method of claim 18, wherein the photo-cleavable linker is irradiated for about 1 minutes to about 15 minutes.

22-104. (canceled)
